(19) United States
(12) Patent Application Publication
(10) Pub. No.: US 2026/0259198 A1
(43) Pub. Date: Sep. 3, 2026

(54) AUTOFLUORESCENCE IMAGING REVEALS THE IMPACT OF CRYOPRESERVATION ON T CELL METABOLISM

(71) Applicant: Wisconsin Alumni Research Foundation, Madison, WI (US)

(72) Inventors: Melissa Skala, Madison, WI (US); Dan Pham, Madison, WI (US)

(21) Appl. No.: 19/067,044

(22) Filed: Feb. 28, 2025

(51) Int. Cl. G01N 33/50

(52) U.S. Cl. CPC G01N 33/5091

(57) ABSTRACT

Systems and methods for determining a processing readiness state of T cells are disclosed. The system includes as autofluorescence image sensor, a processor, and a non-transitory computer-readable memory. The methods include obtaining a T cell from a subject, the T cell having been frozen and thawed, stimulating the T cell, and measuring at least one metabolic indicator within the T cell at a plurality of timepoints based on autofluorescence measurements, analyzing the measurements of the at least one metabolic indicator to characterize a health state of the T cell at each of the plurality of timepoints. The T cell is determined to be ready for processing by comparing the health state of the T cell at each of the plurality of timepoints to a predetermined metabolic state.

### STATEMENT REGARDING FEDERALLY SPONSORED RESEARCH

This invention was made with government support under CA278051 awarded by the National Institutes of Health and under 1648035 awarded by the National Science Foundation. The government has certain rights in the invention.

### BACKGROUND

Cryopreservation plays a central role for several cell therapies including Chimeric Antigen Receptor (CAR) or engineered T cell receptor (TCR) therapy. The cryopreservation protocol, including variables such as cryoprotectant agents, cooling and thawing rate, can have an effect on recovery of cell viability and function upon thawing. Subsequently, T cell activation and efficacy can be impacted by cryopreservation; thus, it is important to characterize the impact of cryopreservation on T cells.

While several bioassays exist for T cell activation assessments, most of them are not suitable for frequent analysis of the same culture over time. What is needed is a way to continuously monitor T cells in real time throughout a post-thaw window to determine changes in the cells and the effects of cryopreservation on cell health and activation response.

### SUMMARY

In one aspect, the present disclosure provides a method of determining a processing readiness state of a T cell. The method includes obtaining a T cell from a subject, the T cell having been frozen and thawed. The method further includes stimulating the T cell, measuring at least one metabolic indicator within the T cell at a plurality of timepoints, analyzing the measurements of the at least one metabolic indicator to characterize an activation response state of the T cell at each of the plurality of timepoints and determining whether the T cell is ready for processing by comparing the activation response state of the T cell at each of the plurality of timepoints to a predetermined metabolic state, where the difference between the predetermined metabolic state and the activation response state of the T cell provides the processing readiness state of the T cell.

In another aspect, the present disclosure provides a system for determining a processing readiness state of a T cell. The system includes a spectrometer configured to obtain a plurality of measurements of at least one metabolic indicator within the T cell at a respective plurality of timepoints after stimulating the T cell at an initial time, the T cell having been frozen and thawed before the initial time, a processor in electronic communication with the spectrometer, and a non-transitory computer-readable medium accessible to the processor and having stored thereon instructions. When executed by the processor, the instructions cause the processor to: analyze the plurality of measurements of the at least one metabolic indicator to characterize an activation response state of the T cell at each timepoint, compare the activation response state of the T cell at each timepoint to a predetermined metabolic state corresponding to a processing readiness state of the T cell, and signal a user that the T cell is in the processing readiness state.

In another aspect, the present disclosure provides a method for developing a cryopreservation protocol for a T cell product. The method includes obtaining a T cell product from a subject, freezing the T cell product, thawing the T cell product, stimulating the T cell product by contacting the T cell product with an antibody reagent or an antigen reagent, obtaining a plurality of measurements of at least one metabolic indicator within the T cell product at a plurality of timepoints, analyzing the plurality of measurements of the at least one metabolic indicator to determine an activation response state of the T cell product at each of the plurality of timepoints, and comparing the activation response state of the T cell product at each of the respective plurality of timepoints to a predetermined metabolic state, where the metabolic indicator comprises at least one of a NAD(P)H fluorescence lifetime (τₘ, τ₁, or τ₂), a NAD(P)H fluorescence amplitude component (α₁ or α₂), a FAD fluorescence lifetime (τₘ, τ₁, τ₂), a FAD fluorescence amplitude component (α₁ or α₂), cytoplasm size, or cell size, and where measuring the at least one metabolic indicator within the T cell comprises at least one of: obtaining a plurality of autofluorescence measurements from the T cell, or exciting the T cell using excitation light, and obtaining at least one of photon counts/intensity or fluorescence lifetimes from at least one of NAD(P)H or FAD stimulated by excitation light in the T cell.

In another aspect, the present disclosure provides a method of determining a processing readiness state of a T cell, including obtaining a T cell from a subject, the T cell having been frozen and thawed, measuring at least one metabolic indicator within the T cell at a plurality of timepoints, analyzing the measurements of the at least one metabolic indicator to characterize a health state of the T cell at each of the plurality of timepoints, and determining whether the T cell is ready for processing by comparing the health state of the T cell at each of the plurality of timepoints to a predetermined state, where the difference between the predetermined state and the health state of the T cell provides a processing readiness state of the T cell.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 is a flowchart illustrating a method, in accordance with an aspect of the present disclosure.

FIG. 2 is a flowchart illustrating a method, in accordance with an aspect of the present disclosure.

FIG. 3 is a block diagram of a device, in accordance with an aspect of the present disclosure.

FIG. 4 shows that cryopreserved T cells underwent significant metabolic changes upon thawing. (A) Representative NAD(P)H mean lifetime (NAD(P)H τₘ) images of donor-matched fresh (top) and frozen (bottom) CD3 T cells throughout 4.5-hour imaging time course. (B-E) Quantification of NAD(P)H τₘ and free NAD(P)H proportion (NAD(P)H α₁) of fresh (B-C) or frozen (D-E) T cells. n=299-537 cells/condition/time point across 3 biologically independent donors. Dots represent individual cells, color coded by condition (fresh or frozen) and donor. Two-sided non-parametric Kruskal-Wallis test with Dunn's posthoc test for multiple comparison against corresponding optical metabolic imaging (OMI) measurements at the 0.5-hour time point. (F-G) Glass's Δs to quantify effect sizes of changes over time in (F) NAD(P)H τₘ and (E) NAD(P)H α₁ of fresh and frozen T cells, with respect to the corresponding OMI measurements at the 0.5-hour time point. Glass's Δ was reported for individual donors as well as the average values across 3 donors. Left 4 columns: fresh T cells, right 4 columns: frozen T cells. Scale bar is 50 μm. Bars are mean±standard deviation. * p<0.05, ** p<0.01, **** p<0.0001.

FIG. 5 shows that fresh and frozen T cells displayed distinct metabolic response towards activating stimulus. (A) Representative NAD(P)H τₘ images of donor-matched fresh (top) and frozen (bottom) T cells upon activation. Frozen T cells were stimulated immediately after thawing. (B-E) Quantification of NAD(P)H τₘ and NAD(P)H α₁ of fresh (B-C) or frozen (D-E) stimulated T cells. n=296-618 cells/condition/time point across 3 biologically independent donors. Two-sided non-parametric Kruskal-Wallis test with Dunn's posthoc test for multiple comparison against corresponding OMI measurements at the 0.5-hour time point. (F-G) Glass's Δs to quantify effect sizes of changes over time in (F) NAD(P)H τₘ and (E) NAD(P)H α₁ of fresh and frozen stimulated T cells, with respect to the corresponding OMI measurements at the 0.5-hour time point. Glass's Δ was reported for individual donors as well as the average values across 3 donors. Left 4 columns: fresh T cells, right 4 columns: frozen T cells. Scale bar is 50 μm. Bars are mean±standard deviation. * p<0.05, ** p<0.01, **** p<0.0001.

FIG. 6 shows the cryopreservation delayed and diminished activation response in frozen T cells upon thawing. (A-D) Quantification of NAD(P)H τₘ and NAD(P)H α₁ of fresh (A-B) or frozen (C-D) T cells from 3 independent donors. Lines represent donor averages, color coded by condition (fresh or frozen) and donor with line patterns based on activation status (dashed line: quiescent, solid line: stimulated). n=296-618 cells/condition/timepoint across 3 donors. ANOVA with three main factors: donor (donor 1, 2, and 3), cryopreservation status (fresh and frozen), and activation status (quiescent and stimulated). Tukey posthoc test was used to determine statistical significance for multiple comparisons between quiescent and stimulated groups at each time point. (E-F) Glass's Δs to quantify effect size of activation on (E) NAD(P)H τₘ and (F) NAD(P)H α₁ of fresh and frozen T cells over time, with respect to corresponding quiescent group at each time point. (G-H) Uniform Manifold Approximation and Projection (UMAP) of 11 OMI parameters (NAD(P)H and FAD τₘ, τ₁, τ₁, α₁, α₂, and cell size) of fresh T cells from 3 donors, color coded by (G) time and (H) activation status. n=4605 cells. (I-J) UMAP of 11 OMI parameters of frozen T cells from 3 donors, color coded by (I) time and (J) activation status. n=3456 cells. Bars are mean±standard deviation. *** p<0.001, **** p<0.0001.

FIG. 7 shows that the activation response of frozen T cells was recovered after 48 hours of activation post-thaw. (A) Representative NAD(P)H τₘ images of fresh and frozen quiescent and stimulated T cells at 48 hours after activation. (B-C) Quantification and (D) comparison of NAD(P)H τₘ of (B) fresh and (C) frozen quiescent and stimulated T cells at 48-hour time point from 3 donors. (E-F) Quantification and (G) comparison of NAD(P)H α₁ of (E) fresh and (F) frozen quiescent and stimulated T cells at 48-hour time point. For (B-G) n=36-195 cells/condition/donors. Two-sided non-parametric Kruskal-Wallis test with Dunn's posthoc tests for multiple comparisons between (B, C, E and F) quiescent versus stimulated groups for each donor and (D, G) fresh versus frozen for all 3 donors. (H) Fold expansion through a 7-day expansion time course of fresh and frozen stimulated T cells from 3 donors. Two-way repeated measures ANOVA with two factors: day (day 1, 4, and 7) and cryopreservation status (fresh and frozen). Sidak posthoc test for multiple comparisons between fold expansions of fresh versus frozen T cells at each time point. 3 data points from 3 independent donors were counted as repeated measurements for statistical test. Scale bar is 50 μm. Bars are mean±standard deviation. * p<0.05, **** p<0.0001.

FIG. 8 shows the experimental setup. On day 0, CD3 T cells were isolated from peripheral blood of healthy donors and divided into two groups for cryopreservation or fresh culture overnight. After 24 hours (day 1), cryopreserved T cells were thawed, and donor-matched fresh T cells were also harvested. Both frozen and fresh T cells were stimulated with αCD2/αCD3/αCD28 T cell activator and imaged with OMI every hour up to 4.5 hours. Fresh and frozen quiescent and activated cells were imaged again at 48 hours (day 3). 72 hours post activation (day 4), cells were counted and resuspended in fresh ImmunoCult™ XF T cell expansion media supplemented with 500 U/mL IL-2. Cells were expanded up to day 7, when cell count was performed to determine fold expansion.

FIG. 9 shows the activation response in fresh and frozen T cells throughout 4.5-hour imaging time course. (A-D) Normalized NAD(P)H intensity and cell size of (A-B) fresh or (C-D) frozen quiescent T cells from 3 independent donors. NAD(P)H intensity at each time point was normalized to donor-matched NAD(P)H intensity measurement at the 0.5-hour time point. n=299-537 cells/condition/time point across 3 donors. (E-H) Normalized NAD(P)H intensity and cell size of (E-F) fresh or (G-H) frozen activated T cells from 3 donors. n=296-618 cells/condition/time point across 3 donors. For (A-H) Two-sided non-parametric Kruskal-Wallis test with Dunn's posthoc test for multiple comparison against corresponding OMI measurements at the 0.5-hour time point. (I-L) Comparison of normalized NAD(P)H intensity and cell size between quiescent and activated groups in (I-J) fresh or (K-L) frozen T cells from 3 donors. n=296-618 cells/condition/timepoint across 3 donors. ANOVA with three main factors: donor (donor 1, 2, and 3), cryopreservation status (fresh and frozen), and activation status (quiescent and activated). Tukey posthoc test was used to determine statistical significance for multiple comparisons between quiescent and activated groups at each time point.

FIG. 10 shows that fresh and frozen T cells displayed different metabolic patterns upon activation. (A-E) UMAP based on 11 OMI features of fresh quiescent and activated T cells at (A) 0.5-, (B) 1.5-, (C) 2.5-, (D) 3.5-, and (E) 4.5-hour imaging time point. (F-G) UMAP based on 11 OMI features of fresh (F) quiescent and (G) activated T cells at all imaging time points. n=4605 cells from 3 donors. (H-L) UMAP based on 11 OMI features of frozen quiescent and activated T cells at (H) 0.5-, (I) 1.5-, (J) 2.5-, (K) 3.5-, and (L) 4.5-hour imaging time point. (M-N) UMAP based on 11 OMI features of frozen (M) quiescent and (N) stimulated T cells at all imaging time points. n=4605 cells from 3 donors.

FIG. 11 shows the activation response of fresh and quiescent T cells at 48 hours. (A-B) Quantification and (C) comparison of cell size of (A) fresh and (B) frozen quiescent and stimulated T cells at 48-hour time point from 3 donors. (D-E) Quantification and (F) comparison of redox ratio of (D) fresh and (E) frozen quiescent and stimulated T cells at 48-hour time point. For (A-F) n=36-195 cells/condition/donors. Two-sided non-parametric Kruskal-Wallis test with Dunn's posthoc tests for multiple comparisons between (A, B, D and E) quiescent versus stimulated groups for each donor and (C, F) fresh versus frozen for all 3 donors.

FIG. 12 shows the antigen-specific activation response in frozen T cells. CMV-specific T cells were stimulated with two different activating stimuli: αCD2/αCD3/αCD28 antibody (antibody activation) or HLA-A*02:01 CMV pp65 peptide (CMV-peptide activation) upon thawing. (A-C) Representative NAD(P)H τₘ images of CMV-specific T cells in (A) quiescent, (B) antibody stimulated, or (C) CMV-peptide groups. APC-conjugated HLA-A*02:01 CMV pp65 peptide showed the presence of HLA-A*02:01 restricted CMV specific T cells (red). 0-hour images were collected immediately post-thaw and before activation. (D-F) Quantification of NAD(P)H τₘ of (D) quiescent, (E) antibody stimulated, and (F) CMV-peptide stimulated CMV-specific T cells throughout 5-hour activation time course post-thaw. Only cells stained positive for APC-conjugated HLA-A*02:01 CMV pp65 peptide (red cells in panel (C)) were included in the analysis for the CMV-peptide stimulated group. n=84-1172 cells/condition/time point across two biologically independent CMV-specific T cell batches (replicates) from one donor. Non-parametric Kruskal-Wallis test with Dunn's posthoc test for multiple comparisons against NAD(P)H τₘ measurements at the 0-hour time point. (G) Comparison of NAD(P)H τₘ between antibody stimulated and CMV-peptide stimulated T cells versus quiescent cells. Lines represent averages from two replicates of CMV-specific T cells from one donor. n=84-1172 cells/condition/time point across 2 replicates. Two-way ANOVA with two factors: time (0, 1, 3, 4, 5 hours) and activation status (quiescent, antibody stimulated, and CMV-peptide stimulated). Dunnett's posthoc test for multiple comparisons of activation status (antibody stimulated versus quiescent, and CMV-peptide stimulated versus quiescent) at each time point. (H) Glass's D calculation for effect size of CD3-mediated (left) and antigen-specific (right) activation on NAD(P)H τₘ of cryopreserved CMV-specific T cells upon thawing. (I) Cytokine production by 200,000 CMV-specific T cells within 5 hours post-thaw with no stimulation (blue) or stimulated with αCD2/αCD3/αCD28 (purple) or HLA-matched CMV peptide (pink). n=6 samples/condition across two batches of CMV-specific T cells. Kruskal Wallis test with Dunn's posthoc test for multiple comparisons. Scale bar is 50 μm. Bars are mean±standard deviation. * p<0.05, ** p<0.01, *** p<0.001, **** p<0.0001.

FIG. 13 shows OMI measurements reveal cell death in patients with diffused-large B cell lymphoma (DLBCL) throughout 4.5 hour upon thawing. Cryopreserved T cells from two DLBCL patients were imaged with OMI immediately upon thawing. NAD(P)H τₘ<1000 ps and optical redox ratio <0.5 were used to gate for dead cells. Numbers represented the percentage of viable cells at each timepoint.

FIG. 14 shows cryopreserved T cells from healthy donors retained viability upon thawing. The same dead cell gate (NAD(P)H τₘ<1000 ps and optical redox ratio <0.5) were applied on T cells from healthy donors. Numbers represented percentage of viable cells at each timepoint.

FIG. 15 shows T cells from DLBCL patients displayed impaired activation response upon stimulation post-thaw. Quantification of NAD(P)H τₘ (A-B) and NAD(P)H α₁ (C-D) from quiescent and stimulated viable T cells from two DLBCL patients throughout 4.5 hours post-thaw. Viable T cells were gated based on OMI measurements (NAD(P)H τₘ>1000 ps or optical redox ratio >0.5).

### DETAILED DESCRIPTION

Before the present invention is described in further detail, it is to be understood that the invention is not limited to the particular embodiments described. It is also understood that the terminology used herein is only for the purpose of describing particular embodiments and is not intended to be limiting. The scope of the present invention will be limited only by the claims. As used herein, the singular forms “a”, “an”, and “the” include plural embodiments unless the context clearly dictates otherwise.

Certain processes as disclosed herein allow assessment of cell health. Cell health can be indicated by characteristics or functions including, but not limited to, viability, proliferation, cytotoxicity, membrane integrity, mitochondrial function, oxygen transport into the cell, waste clearance, or response to stimuli, including activation stimuli. Factors affecting cell health can include environmental or genetic factors, or exposure to drugs or microbes. As used herein, cell health refers to characteristics or functioning of cell processes as measured with respect to a baseline function. The baseline function can be a typical or average function, or the function in the absence of a factor.

Structures, devices, and methods relating to monitoring cell health of cryopreserved T cells after thawing are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, the term “T cell” refers to cells that are CD45+ and CD3+.

As used herein, “cell size” refers to a measured geometric area of a cell of interest as determined by analyzing an acquired image of the cell of interest.

As used herein, “nucleus size” refers to a measured geometric area of a nucleus of a cell of interest as determined by analyzing an acquired image of the cell of interest.

As used herein, “cytoplasm size” refers to the “cell size” minus the “nucleus size.”

As used herein, the term “memory” includes a non-volatile medium, e.g., a magnetic media or hard disk, optical storage, or flash memory; a volatile medium, such as system memory, e.g., random access memory (RAM) such as DRAM, SRAM, EDO RAM, RAMBUS RAM, DR DRAM, etc.; or an installation medium, such as software media, e.g., a CD-ROM, or floppy disks, on which programs may be stored and/or data communications may be buffered. The term “memory” may also include other types of memory or combinations thereof.

As used herein, the term “FAD” refers to flavin adenine dinucleotide.

As used herein, the term “NAD(P)H” refers to reduced nicotinamide adenine dinucleotide and/or reduced nicotinamide adenine dinucleotide phosphate.

As used herein, the term “processor” may include one or more processors and memories and/or one or more programmable hardware elements. As used herein, the term “processor” is intended to include any of types of processors, CPUs, GPUs, microcontrollers, digital signal processors, or other devices capable of executing software instructions.

As used herein, the term “redox ratio” or “optical redox ratio” refers to a ratio of NAD(P)H fluorescence intensity to FAD fluorescence intensity; a ratio of FAD fluorescence intensity to NAD(P)H fluorescence intensity; a ratio of NAD(P)H fluorescence intensity to any arithmetic combination including FAD fluorescence intensity; or a ratio of FAD fluorescence intensity to any arithmetic combination including NAD(P)H fluorescence intensity. In certain cases, the redox ratio or optical redox ratio refers to a ratio of NAD(P)H fluorescence intensity to the sum of NAD(P)H and FAD fluorescence intensity.

Autofluorescence endpoints, or metabolic indicators, include photon counts/intensity and fluorescence lifetimes. The fluorescence lifetime of cells can be a single value, the mean fluorescence lifetime, or compromised from the lifetime values of multiple subspecies with different lifetimes. In this case, multiple lifetimes and lifetime component amplitude values are extracted. Both NAD(P)H and FAD can exist in quenched (short lifetime) and unquenched (long lifetime) configurations; therefore, the fluorescence decays of NAD(P)H and FAD are fit to two components. Generally, NADH and FAD fluorescence lifetime decays are fit to a two component exponential decay, I(t)=α₁e^{-t/τ1}+α₂e^{-t/τ2}+C, where I(t) is the fluorescence intensity as a function of time, t, after the laser pulse, α₁ and α₂ are the fractional contributions of the short and long lifetime components, respectively (i.e., α₁+α₂=1), τ₁ and τ₂ are the short and long lifetime components, respectively, and C accounts for background light. However, the lifetime decay can be fit to more components (in theory any number of components, although practically up to ~5-6) which would allow quantification of additional lifetimes and component amplitudes. By convention, lifetimes t and amplitudes a are numbered from short to long, but this notation could be reversed. A mean lifetime τₘ can be computed from the lifetime components, (τₘ=α₁τ₁+α₂τ₂ . . . ). Fluorescence lifetimes and lifetime component amplitudes can also be approximated from frequency domain data collection and analysis and gated cameras/detectors. For gated detection, α₁ could be approximated by dividing the detected intensity at early time bins by later time bins. Alternatively, fluorescence anisotropy can be measured by polarization-sensitive detection of the autofluorescence, thus identifying free NAD(P)H as the short rotational diffusion time in the range of 100-700 ps.

FAD α₁ refers to the contribution of bound FAD and is the shortest lifetime that is not dominated (i.e., greater than 50%) by instrument response and/or scattering. FAD α₁ is the contribution associated with FAD lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

FAD τ₁ refers to the bound FAD lifetime and is the shortest lifetime that is not dominated (i.e., greater than 50%) by instrument response and/or scattering. FAD τ₁ is the FAD lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

FAD τ₂ refers to the free FAD lifetime and is the longest lifetime that is not dominated (i.e., greater than 50%) by instrument response and/or scattering. FAD τ₂ is the FAD lifetime values from 1000-4000 ps, from 1000-3000 ps, or from 1500-3000 ps. For clarity, a claim herein including features related to a “longest” lifetime cannot be avoided by defining the lifetime values to include a sacrificial shortest lifetime that is dominated by instrument response and/or scattering.

### Methods

This disclosure provides a variety of methods. It should be appreciated that various methods are suitable for use with other methods. Similarly, it should be appreciated that various methods are suitable for use with the systems described elsewhere herein. When a feature of the present disclosure is described with respect to a given method, that feature is also expressly contemplated as being useful for the other methods and systems described herein, unless the context clearly dictates otherwise.

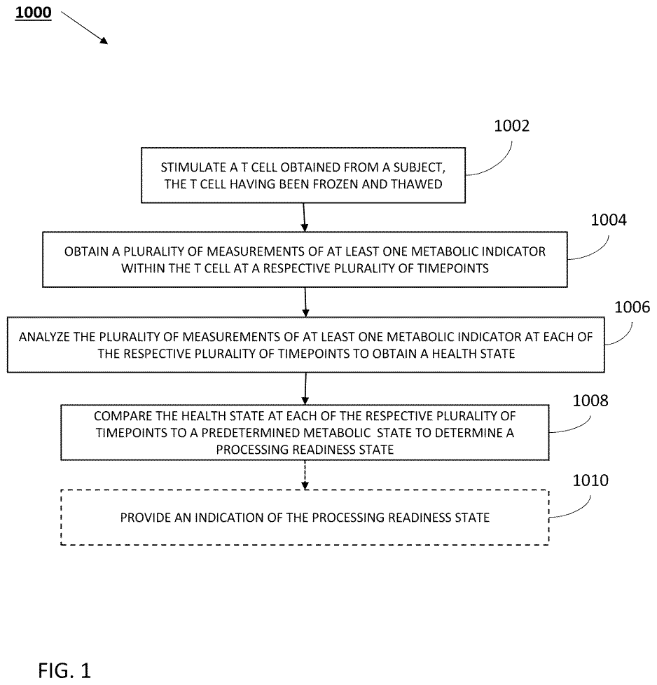

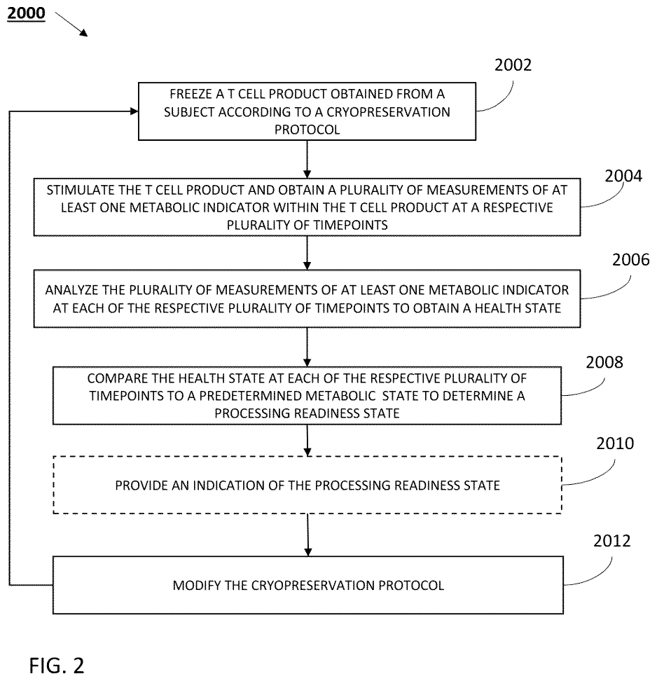

Referring to FIG. 1, the present disclosure provides a method 1000 for determining a processing readiness state of a T cell in accordance with some embodiments of the disclosed subject matter. The processing readiness state of the T cell can be related to the cell health, as indicated by the cell metabolic state, viability, proliferation, cytotoxicity, membrane integrity, mitochondrial function, oxygen transport, waste clearance, or response to stimuli.

At process block 1002, a T cell obtained from a subject is optionally stimulated, where the T cell was frozen and thawed. In some embodiments, the T cell can be stimulated by contacting the T cell with an antibody reagent. For example, the antibody reagent can include soluble antibody complexes that bind to and cross-link CD3, CD28, and CD2 cell surface ligands, (e.g., ImmunoCult™ (αCD2/αCD3/αCD28)), magnetic beads coupled with a combination of anti-human CD3 and anti-human CD28 antibodies (e.g., Dynabeads™ &CD3/αCD28), or a polymeric matrix that contains iron oxide and humanized CD3 and CD28 agonists attached to the matrix (e.g., TransAct™ (αCD3/αCD28)). In other embodiments, the T cell can be stimulated by an antigen reagent. For example, the antigen reagent can be an HLA-matched peptide such as CMV pp65 peptide. In certain embodiments, a subset of a batch of frozen T cells can be stimulated immediately after thawing, and their activation response can be used as an indicator of the processing readiness of the batch of frozen T cells. Other simulation reagents include interleukin 2 (IL2), phorbol 12-myristate 13-acetate (PMA), a phorbol ester, which is a potent activator of protein kinase C (PKC), and ionomycin, a Ca²⁺ ionophore used to raise intracellular Ca²⁺ levels, and to activate T cells. In some embodiments, the T cell is not stimulated.

At process block 1004, a plurality of measurements of at least one metabolic indicator within the T cell can be obtained at a respective plurality of timepoints. Measuring the at least one metabolic indicator within the T cell can include obtaining a plurality of autofluorescence measurements from the T cell. The autofluorescence measurements can be obtained by exciting the T cell using excitation light, and obtaining at least one of photon counts, photon intensity or fluorescence lifetimes from at least one of NAD(P)H or FAD stimulated by excitation light in the T cell. The metabolic indicators can include a NAD(P)H fluorescence lifetime (τₘ, τ₁, or τ₂), a NAD(P)H fluorescence amplitude component (α₁ or α₂), a FAD fluorescence lifetime (τₘ, τ₁, τ₂), or a FAD fluorescence amplitude component (α₁ or α₂). The metabolic indicator can also include cytoplasm size, or cell size as measured using an optical measuring tool.

The at least one metabolic indicator can be determined at a range of timepoints. For example, the timepoints can be every 5 minutes, every 10 minutes, every 15 minutes, every 30 minutes, or every hour. The measurements can be made periodically for up to 1 hour, 2 hours, 3, hours, 4 hours, 5 hours, 6 hours, 12 hours, 18 hours, 24 hours, 36 hours, 48 hours, or 72 hours after thawing and stimulation.

At process block 1006, the plurality of measurements of the at least one metabolic indicator can be analyzed to identify a health state of the T cell. In some embodiments, health state can be a viability state of the T cell. In some embodiments, health state can be a membrane integrity state of the T cell. In other embodiments, health state can be an activation response state of the T cell. In some embodiments, analysis of the metabolic indicator data can include correlating the measurements to the timepoint. Analysis of the metabolic indicator data can include use of a dimensional reduction technique. For example, the dimensional reduction technique can be a Uniform Manifold Approximation and Projection (UMAP), which is used in machine learning and data analysis to visualize high-dimensional data by mapping it onto a lower-dimensional space while preserving the underlying structure and relationships between data points, capturing both local and global patterns within the data. The analysis of the at least one metabolic indicator at a timepoint provides the health state of the T cell at the timepoint.

At process block 1008, the health state of the T cell at each of the plurality of timepoints is compared to a predetermined metabolic state of the T cell to determine the processing readiness state of the T cell. The difference between the predetermined metabolic state and the health state of the T cell provides the processing readiness state of the T cell. For example, the viability state of the T cell can be compared to predetermined threshold values of the metabolic indicators for viability where the processing readiness state of the T cell can be classified as either a desired “viable” state or a “not viable” state. Similarly, the membrane integrity state of the T cell can be compared to the predetermined threshold values of the metabolic indicators for membrane integrity where the processing readiness state of the T cell can be classified as either a desired “intact membrane” state or a “leaky membrane” state. The mitochondrial function of the T cell can be compared to the predetermined threshold values of the metabolic indicators for metabolic function where the processing readiness state of the T cell can be classified as either a desired “functional mitochondria” state or a “dysfunctional mitochondria” state.

In another example, the activation response state of the T cell at each of the plurality of timepoints can be compared to the predetermined metabolic state of the T cell determined from a quiescent T cell that has been obtained from a subject, frozen and thawed. After thawing, the at least one metabolic indicator can be measured within the quiescent T cell at a plurality of timepoints. The timepoints can correspond to the timepoints of the measurements of the T cell. Additionally, and alternatively, the predetermined metabolic state can be determined from a fresh T cell, where the T cell has never been frozen. The at least one metabolic indicator can be measured within the quiescent T cell or the fresh T cell at a plurality of timepoints. The measurements of the at least one metabolic indicator within the quiescent T cell or the fresh T cell can be analyzed at each of the plurality of timepoints to provide the predetermined metabolic state. The processing readiness state can be determined by comparing the metabolic states of quiescent T cells and stimulated T cells at the same timepoint. The difference between the predetermined metabolic state and the activation response state of the T cell provides the processing readiness state of the T cell. The processing readiness state of the T cell can be a desired state (e.g., “ready”), an undesired state (e.g., “not ready”), or any intermediate state.

At optional process block 1010, an indication of the processing readiness state of the T cell can be provided. The indication can be provided at the timepoint when the metabolic state of the T cell reaches a desired state. Additionally, and alternatively, the indication can be provided at some or all of the timepoints prior and up to the T cell reaching a desired state.

The processing readiness state of T cells is a metabolic state that can be correlated to the cell health (e.g., viability, membrane integrity, or other function) of the T cell. Additionally, and alternatively, the processing readiness state of T cells can be a metabolic state that can be used to determine whether the T cell is ready for further processing. Differences in the metabolic states between a quiescent T cell and frozen, stimulated T cells at the same timepoint defines the readiness of the frozen T cells to respond to activating stimuli, such as StemCell™ αCD2/αCD3/αCD28, TransAct™ αCD3/αCD28, Dynabeads™ αCD3/αCD28 or any other antibody or antigen reagents that can induce activation response in T cells. The determination can be made by comparison of time-correlated metabolic indicator data for frozen and thawed stimulated T cells to a predetermined metabolic state, which is determined from time-correlated metabolic indicator data for frozen and thawed quiescent T cells or fresh T cells that have not been frozen and thawed. Additionally, the method 1000 can determine the time needed for frozen and thawed T cells to reach a desired processing readiness state, as the metabolic indicator data is time-correlated.

Referring now to FIG. 2, the present disclosure provides a method 2000 for developing a cryopreservation protocol for a T cell product. In brief, developing a cryopreservation protocol can include observing the effects of the cryopreservation protocol on the processing readiness state of the T cell product, and modifying the cryopreservation protocol with the goal of improving the processing readiness state of the T cell product after cryopreservation. Improvements can include reducing the time it takes for a T cell product to reach a desired processing readiness state, viability state, or increasing the magnitude of the metabolic response to stimulation in process block 2008, as measured by determining the activation response state of the T cell product.

As shown in FIG. 2, process block 2002 includes obtaining a T cell product from a subject and freezing the T cell product according to a cryopreservation protocol. The general steps of cryopreservation protocols are known to those skilled in the art. A person having ordinary skill in the art has knowledge of suitable cryopreservation protocols and the present disclosure is not intended to be bound by one specific implementation of a cryopreservation protocol. The cryopreservation protocol can include, but is not limited to, cell manipulation and handling procedures, freezing procedures, introduction of cryoprotectant additives, and thawing procedures.

Freezing the T cell product can include cooling the T cell product at a rate of between about −0.5° C./min and about −3° C./min to a final temperature. The final temperature can be between 0° C. and −196° C. For example, the final temperature can be below 0° C., below −10° C., below −20° C., below −80° C., or below −130° C. Thawing the T cell product can include warming the T cell product at a rate of between about +0.5° C./min and about +100° C./min to a final thaw temperature, where the final thaw temperature is between 0° C. and 40° C.

The cryopreservation protocol can include addition of a cryoprotectant to the T cell product. The cryoprotectant is a substance that protects cells from damage during freezing. In some examples, the cryoprotectant can be DMSO, glycerol, 2-methyl-2,4-pentanediol (MPD), propylene glycol, or heavy water. In other examples, the cryoprotectant can be a cryoprotective peptide such as fetal bovine serum (FBS). In further examples, the cryoprotectant can be a cryoprotective saccharide such as sucrose, maltose, glucose, mannitol, trehalose, raffinose. Thawing the T cell product can include, but is not limited to, warming the T cell at 37° C., transferring the T cell, centrifuging the T cell, washing the T cell, and introducing the T cell to a cell medium.

Process blocks 2004-2008 correspond to process blocks 1004-1010 of method 1000. In 2004-2008, the T cell product is thawed and the T cell product can be optionally stimulated by contacting the T cell product with an antibody reagent or an antigen reagent. A plurality of measurements of at least one metabolic indicator within the T cell product at a plurality of timepoints is obtained, and the plurality of measurements of the at least one metabolic indicator is analyzed to determine the processing readiness state of the T cell product. For example, a viability state or an activation response state of the T cell product at each of the plurality timepoints can be determined. At process block 2008, the state of the T cell product at each of the respective plurality of timepoints is compared to the predetermined metabolic state to provide the processing readiness state. At process block 2010, an indication of the processing readiness state (e.g., ready or not ready, viable or not viable) can be provided to a user.

At process block 2012 the cryopreservation protocol can be modified based on the indication of the processing readiness state. For example, if the T cell product does not reach a desired processing readiness state as compared to the predetermined metabolic state, at least one aspect of the cryopreservation protocol can be modified. For example, one of the cryoprotectant, the cooling rate, or the final temperature can be modified.

### Systems

This disclosure also provides systems. The systems can be suitable for use with the methods described herein. When a feature of the present disclosure is described with respect to a given system, that feature is also expressly contemplated as being combinable with the other systems and methods described herein, unless the context clearly dictates otherwise.

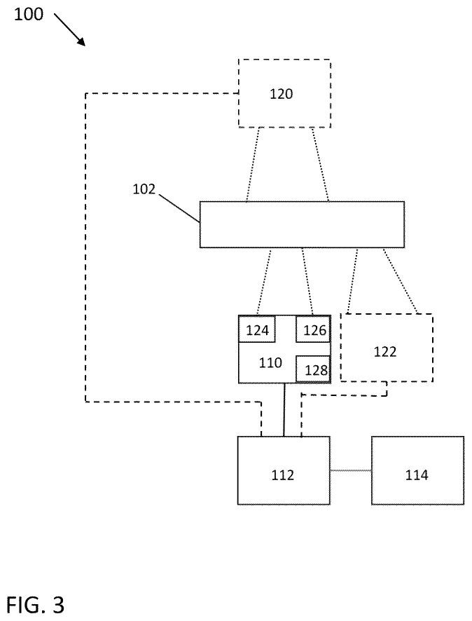

Referring to FIG. 3, the present disclosure provides a system 100 for determining the processing readiness state of a T cell. The system 100 can includes devices, spectrometers, optical and electronic components for a time-resolved autofluorescence decay measurements of cells as described in U.S. Pat. No. 11,410,440, U.S. patent application Ser. Nos. 17/322,367, and 18/397,844, each of which is incorporated herein in its entirety.

According to an aspect of the disclosure herein, the system 100 includes a cell analysis platform 102. The cell analysis platform 102 can be configured to house cells for an extended period of time. For example, the platform 102 can include an incubator designed to fit on the stage of the microscope, allowing for precise temperature, humidity, and gas control of a cell sample while it is being observed under live cell imaging conditions.

The device 100 includes a processor 112 in electronic communication with the spectrometer 110 and a non-transitory computer-readable medium 114, such as a memory, accessible to the processor 112. The non-transitory computer-readable medium 114 can be local to the device 100 or can be remote from the device, so long as it is accessible by the processor 112. In some configurations, the processor 112 can be or otherwise include a field-programmable gate array (FPGA). In configurations where the processor 112 is an FPGA, an additional processor (not shown) may be included to capture images. The time-resolved autofluorescence decay spectrometer 110 includes a pulsed light source 124, a photon-counting detector 126, and photon-counting electronics 128. The time-resolved autofluorescence decay spectrometer 110 can be any spectrometer suitable for acquiring time-resolved autofluorescence decay signals as understood by those having ordinary skill in the optical arts.

Suitable pulsed light sources 124 include, but are not limited to, lasers, LEDs, lamps, filtered light, fiber lasers, and the like. The light source 124 can be pulsed, which includes sources that are naturally pulsed and continuous sources that are chopped or otherwise optically modulated with an external component.

The light source 124 can provide pulses of light having a full-width at half maximum (FWHM) pulse width that is of a duration that is adequate to achieve the spectroscopic goals described herein, as would be appreciated by one having ordinary skill in the spectroscopic arts. In some cases, the FWHM pulse width is at least 1 fs, at least 5 fs, at least 10 fs, at least 25 fs, at least 50 fs, at least 100 fs, at least 200 fs, at least 350 fs, at least 500 fs, at least 750 fs, at least 1 ps, at least 3 ps, at least 5 ps, at least 10 ps, at least 20 ps, at least 50 ps, or at least 100 ps. In some cases, the FWHM pulse width is at most 10 ns, at most 1 ns, at most 900 ps, at most 750 ps, at most 600 ps, at most 500 ps, at most 400 ps, at most 250 ps, at most 175 ps, at most 100 ps, at most 75 ps, at most 60 ps, at most 50 ps, at most 35 ps, at most 25 ps, at most 20 ps, at most 15 ps, at most 10 ps, or at most 1 ps.

The light source 124 can emit wavelengths that are tuned to the absorption of NAD(P)H and/or FAD. In some cases, the wavelength is at least 340 nm, at least 345 nm, at least 350 nm, at least 355 nm, at least 360 nm, at least 365 nm, or at least 370 nm. In some cases, the wavelength is at most 415 nm, at most 410 nm, at most 405 nm, at most 400 nm, at most 395 nm, at most 390 nm, at most 385 nm, or at most 380 nm. In some cases, the wavelength is between 340 nm and 415 nm, between 350 nm and 410 nm, or between 370 nm and 380 nm. In some cases, the wavelength is 375 nm. In some cases, the wavelength is 2 times or 3 times these wavelength values (i.e., the frequency is ½ or ⅓). It should be appreciated that pulsed light sources inherently have some degree of bandwidth, so they are never exactly monochromatic. Thus, references herein to “wavelength” refer to either a wavelength at the peak intensity or a weighted average wavelength. In some cases, the pulsed light source 124 is a UV pulsed diode laser. In some cases, the pulsed light source has a wavelength that is double the peak absorption wavelength of NAD(P)H and/or FAD, with an ultrashort pulse duration, such that fluorescence excitation is achieved through two-photon excitation events, as understood by those having ordinary skill in the optical arts.

The photon-counting detector 126 can be any detector suitably capable of detecting single photons and delivering an analog or digital output representative of the detected photons. Examples of photon-counting detectors 126 include, but are not limited to, a photomultiplier tube, a photodiode, an avalanche photodiode, a single-photon avalanche diode (SPAD), a charge-coupled device, combinations thereof, and the like.

The photon-counting electronics 128 can include electronics understood by those having ordinary skill in the art to be suitable for use with single-photon detectors 126 to produce the decay signals described herein. Examples of suitable photon-counting electronics 128 include, but are not limited to, a field-programmable gate array (FPGA), a dedicated digital signal processor (DSP) with a digitizer and a time-to-digital converter, a time-correlated single photon counting (TCSPC) electronic board with time-to-amplitude and analog-to-digital converter electronics (as implemented by Becker & Hickl, Berlin, Germany), combinations thereof, and the like.

The time-resolved autofluorescence decay spectrometer 110 can be directly (i.e., the processor 112 communicates directly with the spectrometer 110 and receives the signals) or indirectly (i.e., the processor 112 communicates with a sub-controller that is specific to the spectrometer 110 and the signals from the spectrometer 110 can be modified or unmodified before sending to the processor 112) controlled by the processor 112. Time-resolved autofluorescence decay signals can be acquired by known spectroscopic methods. Fluorescence lifetime images can also be acquired by known imaging methods and those acquired images can be used by the systems and methods described herein, as would be understood by those having ordinary skill in the spectroscopic arts. The device 100 can include various optical filters tuned to isolate autofluorescence signals of interest. The optical filters can be tuned to the autofluorescence wavelengths of NAD(P)H and/or FAD. The device 100 can be substantially free of fluorescent labels. The device 100 can be substantially free of immobilizing agents for binding and immobilizing T cells.

The time-resolved autofluorescence decay spectrometer 110 can be configured to acquire the autofluorescence dataset from the detector's 126 electrical output at a repetition rate understood by those having ordinary skill in the spectroscopic arts to be suitable for providing adequate sampling to observe the dynamics disclosed herein. In some cases, the repetition rate can be at least 1 kHz, at least 5 kHz, at least 10 kHz, at least 30 kHz, at least 50 kHz, at least 100 kHz, at least 500 kHz, at least 750 kHz, at least 1 MHz, at least 4 MHz, at least 7 MHz, at least 10 MHz, at least 15 MHz, at least 20 MHz, at least 50 MHz, at least 100 MHz, at least 500 MHz, or at least 1 GHz. In some cases, the repetition rate can be at most 1 THz, at most 800 GHz, at most 500 GHz, at most 250 GHz, at most 150 GHz, at most 100 GHz, at most 70 GHz, at most 50 GHz, at most 25 GHz, at most 15 GHz, at most 10 GHz, at most 6 GHz, at most 2 GHz, at most 1 GHz, at most 750 MHz, at most 500 MHz, at most 400 MHz, at most 250 MHz, at most 175 MHz, or at most 100 MHz. While there can be downside associated with oversampling, in principle the present disclosure can function with as high of a sampling rate as can be achieved with existing technology. The repetition rates identified herein are based on the state of the art at the time the present disclosure was prepared and filed and are not intended to be limiting in the event that future developments facilitate a greater repetition rate.

The pulsed light source 124 can be configured to operate at pulse repetition rates that are adapted to acquire the needed fluorescence lifetime information. The maximum pulse repetition rate is limited by the fluorescence lifetime of the fluorophore of interest. The fluorescence decay must have fully died down by the time the next pulse of light is introduced to the sample in order to avoid ambiguity about the sources of decay signals (i.e., was this particular fluorescent photon initiated by the most recent excitation pulse of light or the one preceding it?). The pulsed light source 124 can have a pulse repetition rate of up to 100 MHz, up to 80 MHz, up to 60 MHz, or up to 40 MHz. The lower limit of the pulse repetition rate is more practical in a sense of reducing the overall sampling time, but theoretically the data can be taken very slowly if there is some reason to do so.

The device 100 can optionally include an optical microscope 120 for acquiring visual images of cells that are located in the cell analysis platform 102. The device 100 can optionally include a cell size measurement tool 122. The cell size measurement tool 122 can be any device capable of measuring the size of cells, including but not limited to, an optical microscope, such as optical microscope 120. In some cases, the optical microscope and the cell size measurement tool 122 are the same subsystem.

In some cases, the time-resolved autofluorescence decay spectrometer 110 and the optical microscope 120 can be integrated into a single optical subsystem. In some cases, the time-resolved autofluorescence decay spectrometer 110 and the cell size measurement tool 122 can be integrated into a single optical subsystem. While some aspects of the methods described herein can operate by not utilizing the cell size as an input to the convolutional neural network, it may be useful to measure the cell size for other purposes.

The time-resolved autofluorescence decay spectrometer 110 can be configured to obtain a plurality of measurements of at least one metabolic indicator within the T cell at a respective plurality of timepoints. The T cell can be stimulated at an initial time, the T cell having been frozen and thawed before the initial time. The T cell can be stimulated by contacting the T cell with at least one of an antibody reagent or an antigen reagent.

The non-transitory computer-readable medium 114 has stored thereon instructions that, when executed by the processor, cause the processor to execute at least a portion of the methods described herein. The instructions, when executed by the processor 112, can further cause the processor to analyze the plurality of measurements of the at least one metabolic indicator to characterize the activation response state of the T cell at each timepoint. The activation response state of the T cell can be characterized based on the analysis of the metabolic indicators from the autofluorescence decay measurements of the T cell at each of the respective plurality of timepoints. For example, the measurements can include obtaining a plurality of values of at least one metabolic indicator at each of the respective plurality of timepoints. The instructions can cause the processor 112 to analyze the plurality of values of the at least one metabolic indicator. For example, the at least one metabolic indicator can be NAD(P)H τₘ and NAD(P)H α₁. Accordingly, the processor 112 can determine the activation response state of the T cell based on analyzing the plurality of values of NAD(P)H τₘ and the plurality of values of NAD(P)H α₁.

The instructions can, when executed by the processor 112, can cause the processor 112 to determine the predetermined metabolic state by measuring at least one metabolic indicator within a quiescent T cell at a plurality of timepoints and analyzing the measurements of the at least one metabolic indicator of the quiescent T cell at each of the plurality of timepoints to provide the predetermined metabolic state. In some embodiments, the processor 112 can be caused by the instructions to identify the predetermined metabolic state based on identifying at least one of a minimum value of the plurality of values of NAD(P)H τₘ of the quiescent T cell or a maximum value of the plurality of values of NAD(P)H α₁ of the quiescent T cell.

The predetermined metabolic state can be stored in the processor or the non-transitory computer-readable medium 114. In some embodiments, the predetermined metabolic state can be imported to the non-transitory computer-readable medium 114 from an external source.

The processor 112 can be caused by the instructions to compare the viability state or the activation response state of the T cell at each timepoint to the predetermined metabolic state to determine the processing readiness state of the T cell. The processor 112 can be further caused by the instructions to signal the processing readiness state of the T cell to a user. For example, the user can receive a signal from the device that the T cell is in a desired processing readiness state, is not yet in a desired readiness state, or an estimate of time to the desired processing readiness state.

In use, the device 100 can be used to monitor the cell health of cryopreserved cells that have been thawed. For example, the device 100 can measure the metabolic state of cryopreserved T cells over time after thawing and indicate to a user when the thawed T cells reach a predetermined state. For example, the device can indicate when the thawed T cells are ready for further processing, such as patient infusion or adoptive T cell therapy manufacturing. Cell therapies can include chimeric antigen receptor (CAR) T therapy, engineered T-cell receptor (TCR) therapy, or tumor-infiltrating lymphocyte (TIL) therapy.

In some cases, a subset of a batch of cryopreserved T cells can be monitored as a representative portion of a batch. When a batch of cryopreserved T cells is thawed, a subset of the batch can be immediately subjected to the activation conditions and the metabolic state measured over time to determine when the cryopreserved T cells reach a particular state of function after they are thawed. When the activation measurement (OMI) gives a positive signal for the subset, the batch of thawed T cells is indicated as ready for use. The thawed T cells can then be used for patient infusion or cell manufacturing. For example, the thawed T cells can be used for chimeric antigen receptor (CAR) T therapy, engineered T-cell receptor (TCR) therapy, or tumor-infiltrating lymphocyte (TIL) therapy. In some cases, the thawed T cells do not need to be stimulated (e.g., patient CAR T cells do not need to contact with antibody/antigen reagents before patient infusion). The ability to detect when the thawed cells reach a ready-for-use state can save time and resources by ensuring the cells are competent when they are administered.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

Cryopreservation is commonly used for long-term preservation of cells, tissues and other biological samples, which involves using low temperature (e.g., −80° C. to −196° C.) to slow down cell metabolism and other functional activity. Cryopreservation plays a central role for several cell therapies including Chimeric Antigen Receptor (CAR) or engineered T cell receptor (TCR) therapy. The complex manufacturing process of these cell therapies requires specialized facilities and expertise, which uses a central manufacturing model that relies on cryopreservation of both the starting materials and final products¹. For instance, the current CAR T manufacturing workflow starts with collection of leukapheresis from patient at the hospital site as the starting material. A patient's leukapheresis product is then cryopreserved and transported to the manufacturing facility, where T cells are isolated upon thawing, followed by T cell activation, CAR gene transfer, and expansion². At the end of the manufacturing process, the CAR T cell product is cryopreserved. This process allows for preservation of the product while necessary quality control testing is performed, and for transportation to the hospital for infusion into patient. Despite ongoing research to define the optimal cryopreservation protocol such as cryoprotectant agents, cooling and thawing rate, the successful recovery of cell viability and function upon thawing remains challenging^{2, 3}. Meanwhile, due to the use of cryoprotectant agents such as dimethyl sulfide (DMSO) that has potential toxic effects on cell viability and effector functions of immune cells⁴, several CAR T cell products mandate infusion within 30 minutes after thawing⁵. Since proper T cell function is related to the efficacy of CAR T cell therapy, it is important to characterize the impact of cryopreservation on T cells within a short time frame post-thaw, particularly concerning T cell activation response, a key function for CAR T manufacturing success and clinical outcome.

For T cell activation, metabolic reprogramming provides the necessary energy and molecules for effector function, but also to shape the epigenetic landscape that determines T cell differentiation fate⁸⁻¹⁰. Cryopreservation has been shown to affect cell viability and metabolism-related proteins while reducing metabolic activity in various mammalian cell models such as embryos and other reproductive cells^{11,12,13}, engineered tissues (osteoblasts¹⁴), and transplanted hepatocytes^{15,16} or pancreatic islets^{17,18} However, the impact of cryopreservation is cell-type dependent¹⁹, and immediate metabolic changes in T cells post-thaw and their implication on early activation response remain unclear.

While several bioassays exist for metabolism and T cell activation assessments, most of them either require destructive and time-consuming sample preparation that is not suitable for frequent analysis of the same culture over time, or do not offer single-cell resolution to characterize heterogeneous cell function.

During CAR T cell manufacturing, T cell activation is induced by general antibodies targeting stimulatory and co-stimulatory receptors on the T cell surface, such as CD3, CD28, and CD2 upon isolation from the cryopreserved leukapheresis product⁶. Activation primes T cells for CAR gene transfer while inducing proliferation to achieve a sufficient CAR T cell dosage, both of which are required for successful CAR T cell manufacturing⁷. Meanwhile, antigen-specific activation of CAR T cells upon infusion into the patient and interaction with target tumor cells initiates cytotoxic functions to eliminate cancer cells, leading to tumor regression and patient treatment response. At either stage, T cell activation is required within a short time window upon thawing of previously cryopreserved products. Therefore, understanding how cryopreservation affects CD3-mediated and antigen-specific activation of T cells upon thawing will allow optimization of cryopreservation protocols that ultimately enhance CAR T cell performance.

To address the challenge of assessing metabolism and T cell activation of cryopreserved cells, we developed optical metabolic imaging (OMI), a label-free non-invasive imaging method that allows assessment of single cell metabolism based on autofluorescent signals from metabolic coenzymes NAD(P)H and FAD^{20,21,22}. NAD(P)H and FAD are an electron donor and acceptor, respectively for various metabolic pathways, including those important for T cell function and activation response such as glycolysis^{23,24}. OMI yields 14 cellular metabolic features or metabolic indicators based on fluorescence intensity and lifetime of NAD(P)H and FAD. Fluorescence lifetime is defined as time taken for a molecule in the excited state to decay back to the ground state and emit a fluorescence signal. Free NAD(P)H self-quenches, resulting in a short fluorescence lifetime of around 400 ps^{25,26}. Upon protein-binding, NAD(P)H undergoes conformational changes, leading to long fluorescence lifetime of around 2.2-2.5 ns^{26,27}. FAD displays reversed trend in fluorescence lifetime, with free FAD having long lifetime and bound FAD being characterized with short lifetime^{24,28}. Therefore, the fluorescence lifetimes of NAD(P)H and FAD are indicative of their binding activity.

Here, the inventors demonstrate use of OMI to determine the impact of cryopreservation on T cell metabolism and activation response, along with implications of cryopreservation for adoptive T cell therapy. Using OMI, the inventors evaluate metabolic response accompanying different modes of T cell activation, including CD3-driven and antigen-specific activation in cryopreserved cells.

### Methods

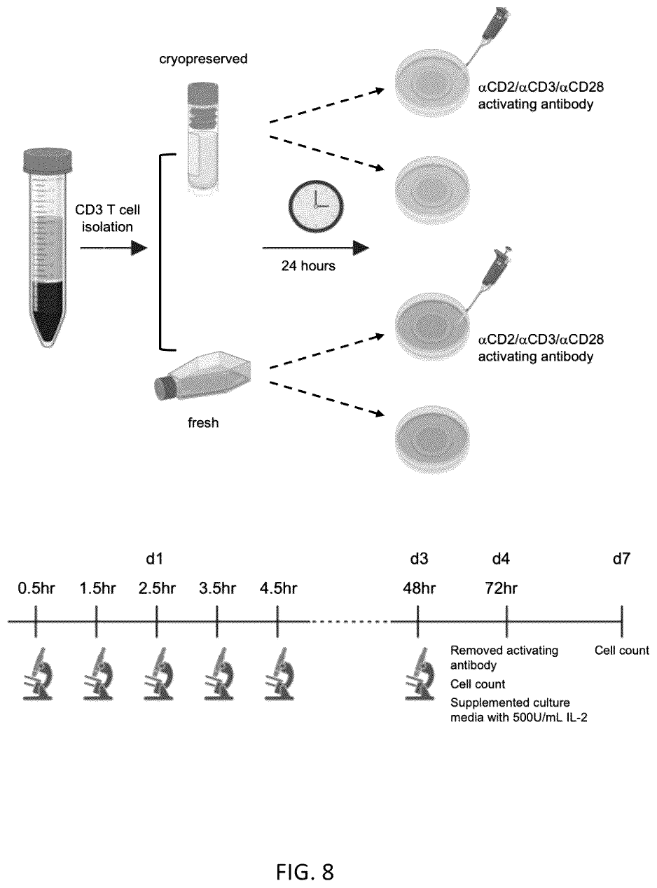

T cell isolation and culture: Healthy donors were recruited and informed consent was collected from all donors under a protocol approved by the Institutional Review Board at the University of Wisconsin-Madison. CD3 T cells were isolated from peripheral blood of healthy donors using RossetteSep Human T cell enrichment cocktail (STEMCELL Technologies) following the manufacturer's protocol. Briefly, peripheral blood was incubated with 50 mL/mL T cell enrichment cocktail, then diluted in PBS+2% Fetal Bovine Serum (FBS) and layered on Lymphoprep density gradient (STEMCELL Technologies) for separation using centrifugation. Isolated T cells were then divided into two groups for fresh culture or cryopreservation. For the fresh culture group, T cells were resuspended in ImmunoCult™ XF T cell expansion medium at 1 million cells/mL and cultured overnight at 37° C., 5% CO₂. For cryopreservation, T cells were resuspended in FBS+10% DMSO at 1 million cells/mL. Cryopreservation vials containing T cells were then encapsulated in a freezing container (CoolCell LX Cell Freezing Container, Corning) to control for freezing rate (−1° C./minute) and transferred to −80° C. freezer to be frozen overnight (FIG. 8).

Thawing and activation of T cells: Cryopreservation vials were submerged in a water bath at 37° C. for 30 seconds. Warm fresh ImmunoCult™ XF T cell expansion medium was then added to the cryopreservation vial one drop at a time. When no ice crystals were visible, all T cells were then transferred into 10 mL of warm fresh ImmunoCult™ XF media, then centrifuged to wash out DMSO residue. T cells were then counted and plated at 200,000 cells in 200 mL of fresh media into a glass-bottom 96 well plate. Immediately upon thawing and plating, frozen T cells were activated with 5 mL of StemCell αCD2/αCD3/αCD28 antibody (FIG. 8).

For the antigen specific experiment, HLA-A*0201 restricted anti CMV T cells were obtained from Celero (item #1049-5147JN21 and item #1049-5085AP21). Upon arrival, T cells were kept in liquid nitrogen until used. T cell thawing was performed following a similar protocol as above. Briefly, RPMI+2% FBS+1% Pen/Strep was warmed up to 37° C., then added by drop into cryopreserved T cell vials. T cells were then washed once with fresh warm media and plated at a density of 1 million cells/mL. Immediately upon thawing, CMV-specific T cells were plated onto 35 mm glass bottom, Poly-D-lysine coated imaging dishes (MatTek) at a concentration of 200,000 cells/200 mL Immunocult™ T cell expansion media. T cells were then stimulated with either 5 mL of StemCell αCD2/αCD3/αCD28 antibody or 1 mL iTAg Tetramer/APC-HLA-A*02:01 CMV pp65 peptide (NLVPMVATV) (MBL International) to generate antibody and CMV activated groups, respectively.

T cell imaging with OMI: 30 minutes after thawing and stimulation, T cells were imaged using OMI for every hour up to 4.5 hours in a stage top incubator (37° C., 5% CO₂) (FIG. 8) as previously described³¹. OMI was performed on a custom-build multiphoton microscope (Ultima, Bruker) using an inverted laser-scanning microscope body (Ti-E, Nikon) equipped with an ultrafast tunable laser source (Insight DS+, Spectsra Physics). Two-photon excitation of NAD(P)H and FAD were performed at 750 nm (2.5 mW) and 890 nm (4.5 mW), respectively, using a 40× water immersion 1.15 NA objective (Nikon) with 2.5× optical zoom. Other imaging parameters include 4.8 μs pixel dwell time, 60 s integration time, and image size of 256×256 pixels. NAD(P)H and FAD emission spectra were collected using GaAsP photomultiplier tubes (H7422, Hamamatsu) using a 440/80 nm and 550/100 nm bandpass filters, respectively. Fluorescence decays of NAD(P)H and FAD were acquired using time-correlated single-photon counting (TCSPC) electronics (SPC 150, Becker & Hickl GmbH) using Prairie View Software (Bruker). APC-conjugated CMV peptide was excited at 980 nm and emission signal was collected using 690/50 nm filter. Fluorescence intensity and lifetime images of NAD(P)H and FAD, together with immunofluorescence images of APC-conjugated CMV peptide, were collected for each field of view (FOV), with 3-5 representative FOVs imaged per condition. The instrument response function was measured using second-harmonic generation signal from urea crystals excited at 890 nm.

Image segmentation and OMI analysis: Fluorescence decay curves collected with TCSPC were fit to a double-exponential decay in SPCImage software to determine pixel-wise lifetimes of free and protein-bound NAD(P)H and FAD. Pixel-wise optical redox ratio was calculated as the normalized ratio between NAD(P)H fluorescence intensity and the sum of NAD(P)H and FAD fluorescence intensity. Individual cell and individual cytoplasm were segmented for CD3-mediated and antigen-specific experiments, respective. CellPose cyto2 model was used for automatic segmentation of whole cells, with manual check by users to ensure accurate masking for each FOV. For cytoplasm segmentation, a customized CellProfiler pipeline was used for manual segmentation of individual nuclei and whole cells in each FOV. The whole cell and cytoplasm masks were applied on the corresponding OMI images to compute mean values of OMI variables for each cell or each cytoplasm. 13 OMI variables were collected and quantified, including: optical redox ratio, fluorescence intensity and mean fluorescence lifetime (τₘ) of NAD(P)H and FAD, their free- and protein-bound lifetime components (τ₁, τ₂) and corresponding fractional contributions (α₁, α₂).

Statistical analysis: Statistical significance among experimental groups was performed in Prism (v10.2.2) or R-Studio (v3.5.3). Two-sided non-parametric Kruskal-Wallis test was used to eliminate bias and assumption of normality in the data set. Dunn's posthoc test were chosen to adjust for multiple comparisons. To determine effect size of metabolic changes within the same treatment condition (eg: frozen quiescent, frozen stimulated, fresh quiescent, or fresh stimulated group), Glass's Δ was calculated as:

$$Glass ' s {Δ}_{ij} = \frac{{μ}_{ij} - {μ}_{i 0.5 hr}}{{σ}_{i 0.5 hr}}$$

where μᵢⱼ is the mean value for the OMI parameter of interest for donor i at time point j, μ_{i 0.5 hr} is the donor-matched mean value of the corresponding OMI parameter at 0.5 hour timepoint, and σ_{i 0.5 hr} is the standard deviation of μ_{i 0.5 hr}. To determine the effect size of activation on T cell metabolism at each time point,

$$Glass ' s {Δ}_{ij} = \frac{{μ}_{{activated}_{ij}} - {μ}_{{quiescent}_{ij}}}{{σ}_{{quiescent}_{ij}}} .$$

Glass's Δ values were reported for individual donors, as well as the averaged values across three donors.

### Results

OMI identifies significant changes in frozen T cell metabolism upon thawing

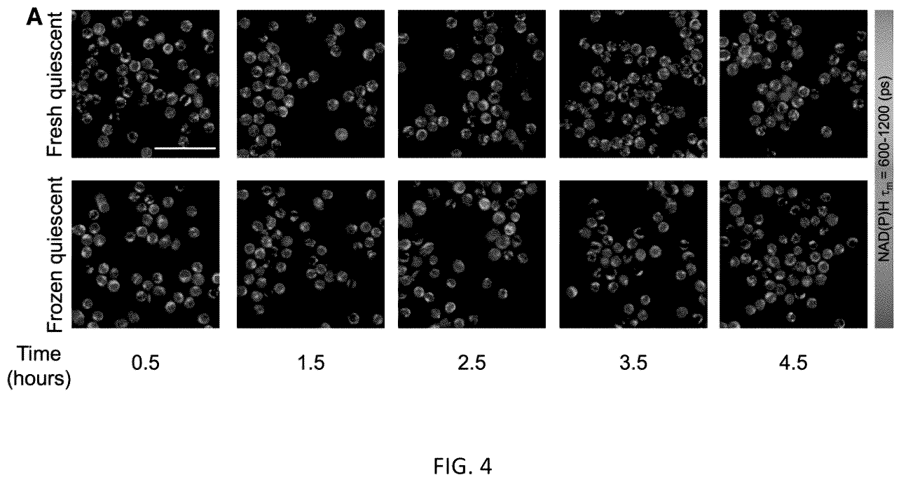

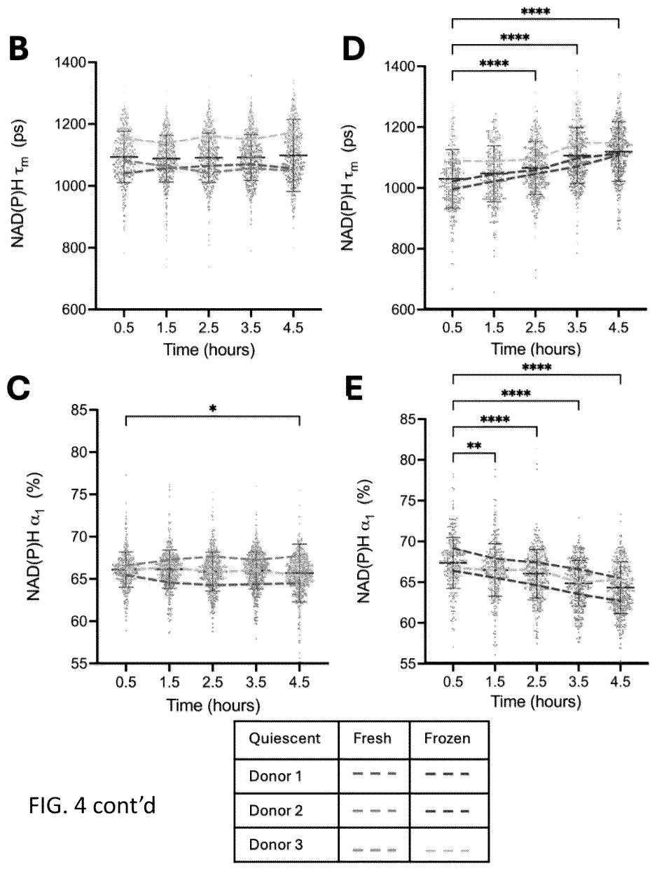

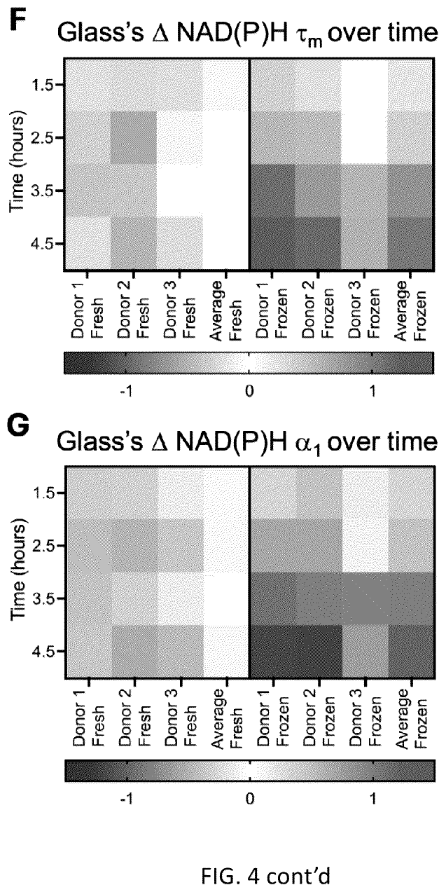

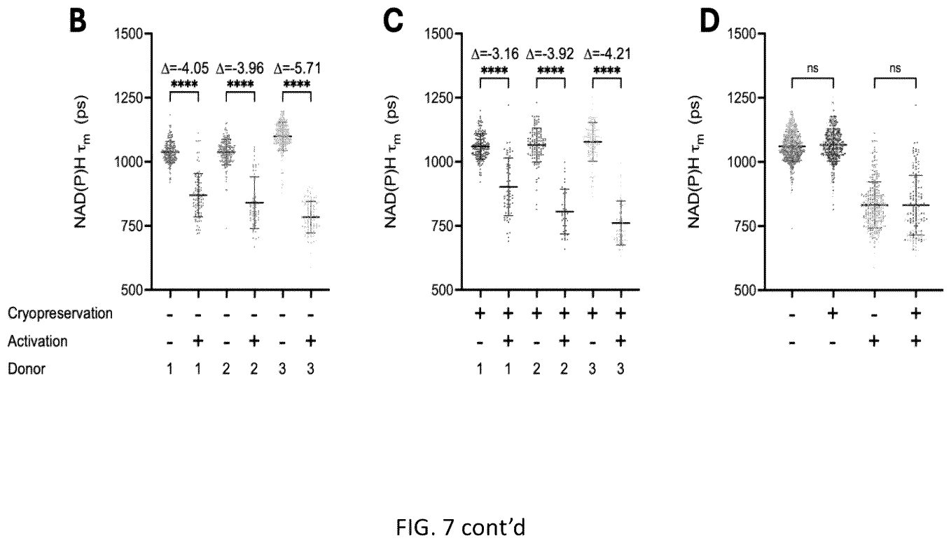

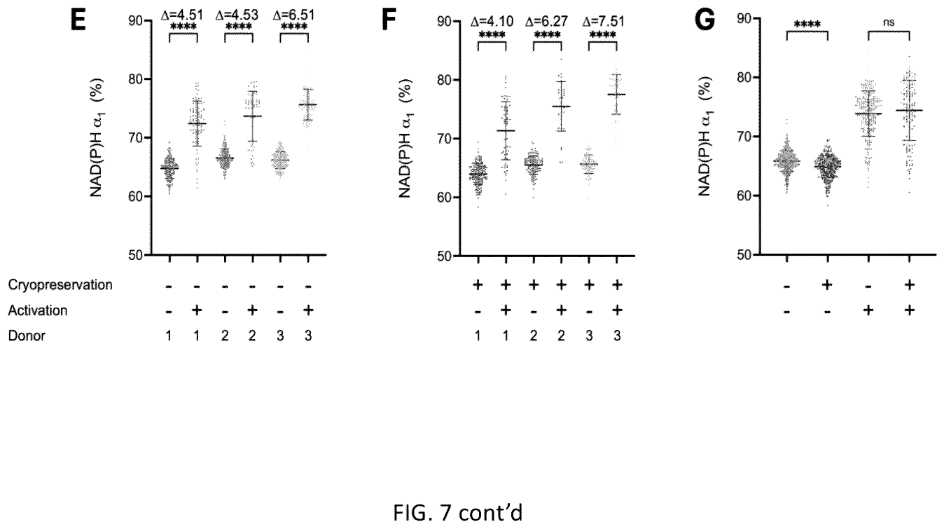

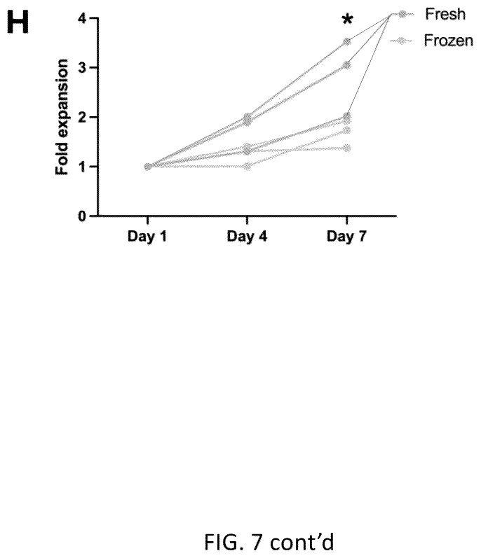

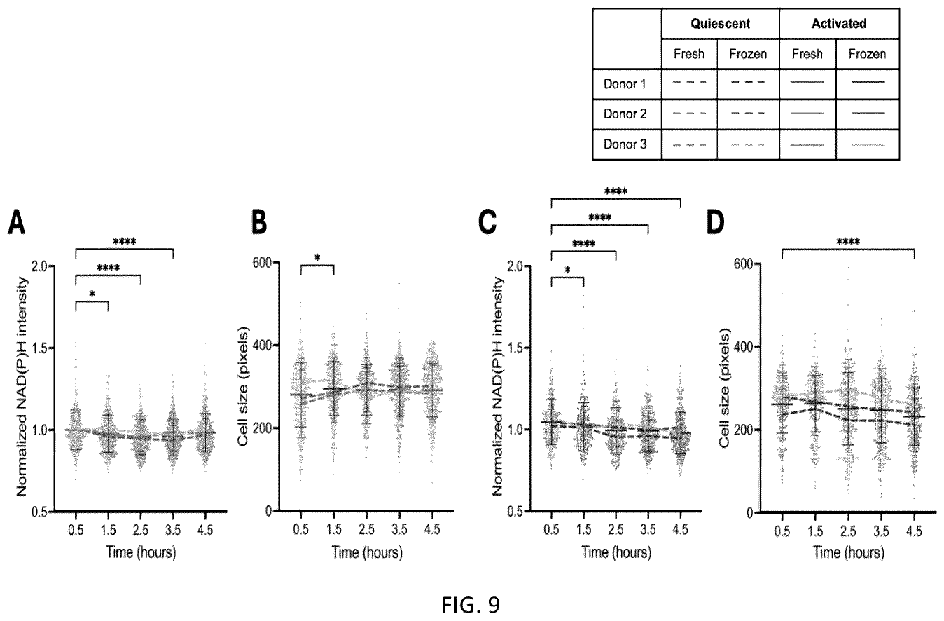

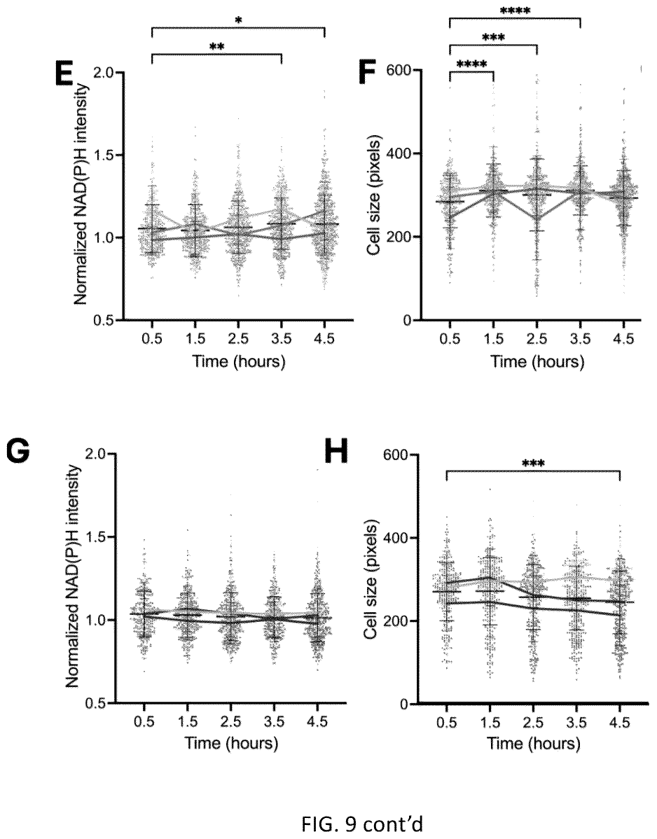

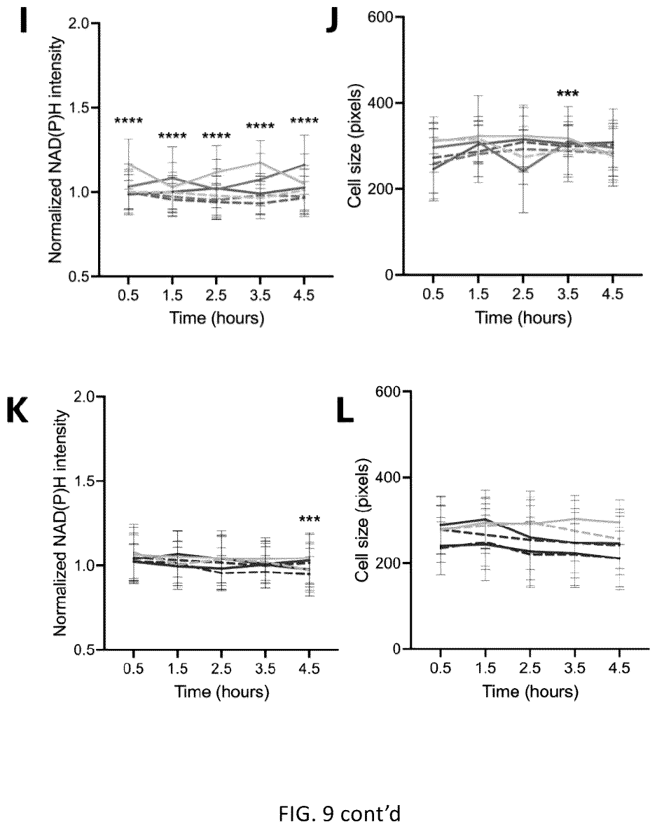

Quiescent CD3 T cells from 3 donors were imaged using OMI every hour upon thawing (FIG. 7A). Throughout the 4-hour imaging time course, fresh quiescent T cells displayed stable metabolism, with no significant changes in NAD(P)H mean lifetime (NAD(P)H τₘ) and a slight increase in the proportion of free NAD(P)H (NAD(P)H α₁) at the 4.5-hour time point (FIG. 4B, 4C). Interestingly, we observed significant and consistent metabolic changes in frozen quiescent T cells upon thawing. Across 3 donors, frozen T cells showed a gradual but significant increase in NAD(P)H τₘ and decrease in NAD(P)H α₁ within the first 4.5 hours post-thaw. Changes in NAD(P)H lifetime parameters in fresh and frozen quiescent T cells were accompanied by decrease in normalized NAD(P)H intensity (FIG. 9A-D). Overall, cell size of both fresh and frozen quiescent T cells remained relatively stable throughout the imaging time course (FIG. 8). To determine the effect sizes of metabolic changes over time, we calculated Glass's Δ at each time point with respect to the 0.5-hour time point (FIGS. 4F, 4G). Glass's Δ revealed a small effect of imaging time on fresh quiescent T cell metabolism. For several OMI parameters, the average effect size across 3 donors at each time point remain within −0.1 to 0.1, further indicating stable metabolism (FIGS. 4F, 4G—left). Meanwhile, we observed large effect of time post-thaw on frozen quiescent T cell metabolism. Over time, Glass's Δ for several OMI parameters of frozen T cells strengthened and reached an average value of greater than 1 (for NAD(P)H τₘ) or smaller than −1 (for NAD(P)H α₁) at the 4.5-hour time point (FIGS. 4F, 4G—right). This indicated that the differences in these OMI parameters exceeded one standard deviation throughout the imaging time course. Overall, our findings suggested that OMI did not affect fresh T cell metabolism, and the use of a stage top incubator was sufficient to stabilize cell metabolism during imaging. Using OMI, we have also determined consistent and significant metabolic changes in frozen T cells, with a shift towards increasing NAD(P)H τₘ and decreasing NAD(P)H α₁ that continued up to 4.5-hour post thaw.

### Fresh and Frozen T Cells Displayed Distinct Metabolic Response to Activation During the First 4.5 Hours

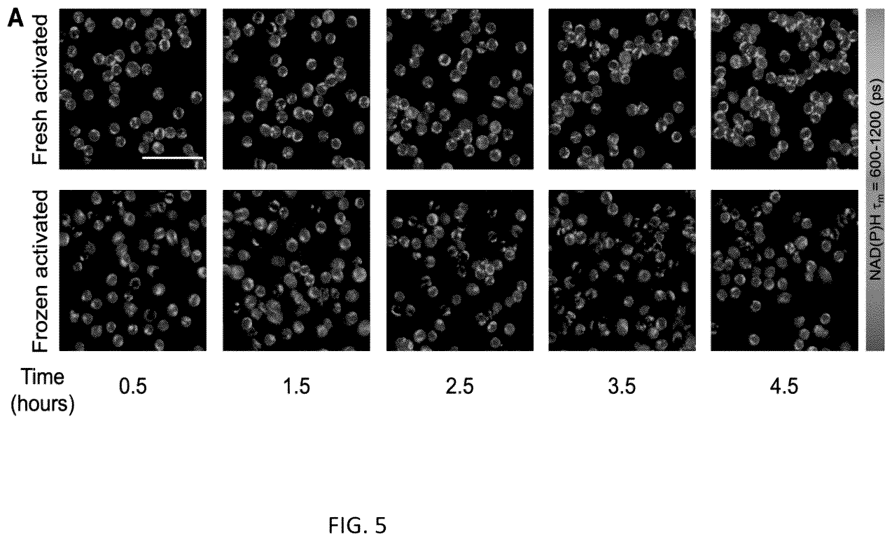

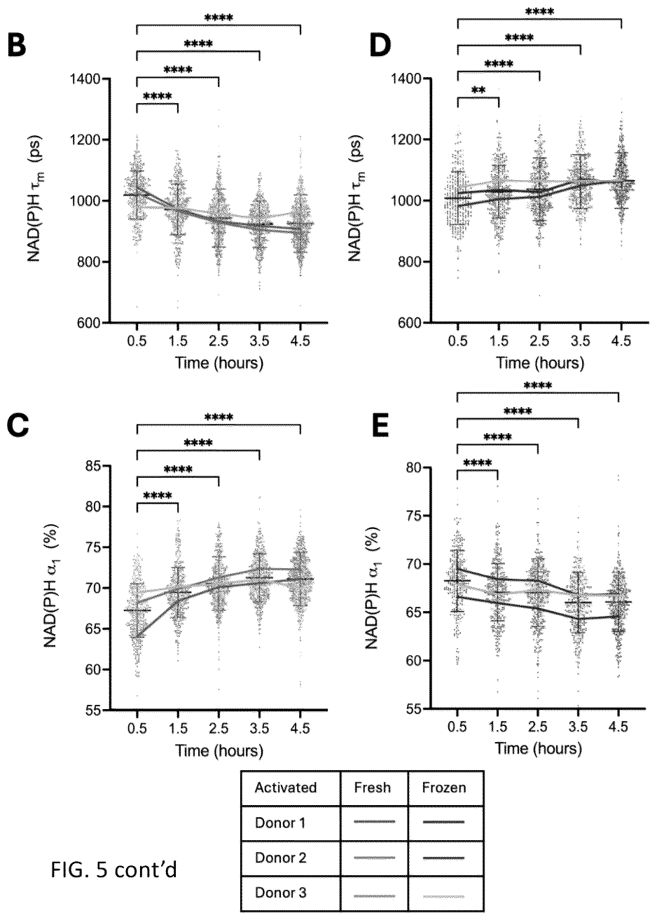

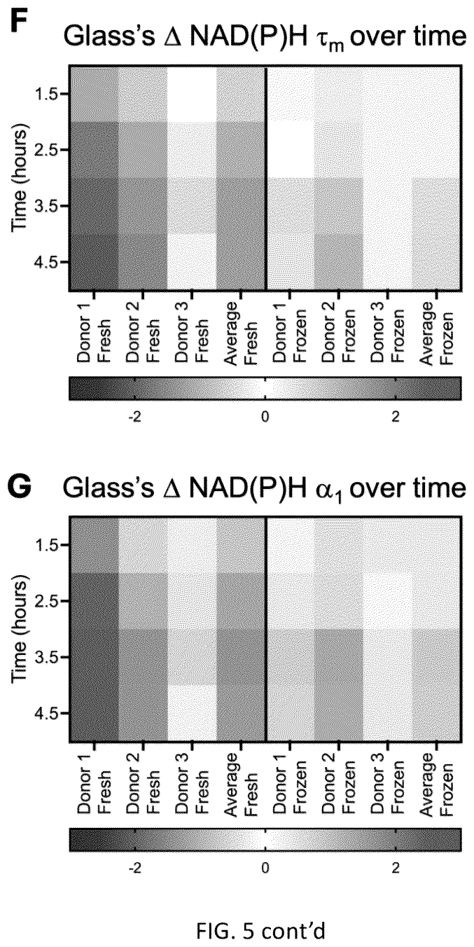

Metabolic reprogramming is crucial for T cell activation to support effector functions and proliferation. Due to significant changes in frozen T cell metabolism upon thawing, we further investigated how cryopreservation impacts T cell activation response using OMI. Frozen T cells were immediately activated with StemCell &CD2/αCD3/αCD28 upon thawing. Similarly, donor-matched fresh T cells were also activated at the same time (FIG. 5A). In fresh T cells, we observed the characteristic metabolic changes, with low NAD(P)H τₘ and high NAD(P)H α₁, following stimulation (FIGS. 5B, 5C). This is consistent with previous studies showing a shift towards glycolysis in activated T cells^{8,9}. These changes in T cell metabolism occurred early and were significant within 1 hour of stimulation. Activation-induced metabolic changes in fresh T cells continued to progress, with decreasing NAD(P)H τₘ and increasing NAD(P)H α₁ observed over time (FIGS. 5B, 5C). Increased cell size was also previously reported as an indicator of T cell activation^{29,32}. During the first 4.5 hours post activation, fresh activated T cells also displayed a slight increase in cell size and normalized NAD(P)H intensity, though not as significant as changes in NAD(P)H lifetime parameters (FIGS. 9E, 9F). We did not, however, observe similar metabolic changes in frozen stimulated T cells (FIGS. 5D, 5E). Across 3 donors, frozen stimulated T cells showed increased NAD(P)H τₘ and decreased NAD(P)H α₁ that were sustained throughout the 4-hour imaging time course. No significant changes in normalized NAD(P)H intensity were observed in frozen stimulated T cells, though we did observe a slight decrease in cell size at the 4.5-hour time point (FIGS. 9G, 9H). These metabolic changes were opposite of donor-matched fresh stimulated T cells (FIGS. 5B, 5C) but were consistent with frozen quiescent T cells (FIG. 7).

Glass's Δ with respect to the 0.5-hour time point further confirmed opposite metabolic responses of donor-matched activated fresh and frozen T cells over time (FIGS. 5F, 5G). Throughout stimulation (0.5- to 4.5-hour), fresh T cells showed large effects in NAD(P)H τₘ and NAD(P)H α₁ that strengthened over time, with average absolute Glass's Δ values exceeding 1.2 at the 4.5-hour time point (FIGS. 5F, 5G—left). These findings demonstrated that T cell metabolism, especially NAD(P)H binding activity, were highly responsive to activating stimulus. Meanwhile, for frozen T cells, we observed weak- to moderate-effect sizes throughout the imaging time course (FIGS. 5F, 5G—right). Interestingly, at 4.5 hours, average Glass's Δ of frozen activated T cells (0.60 for NAD(P)H τₘ and −0.68 for NAD(P)H α₁) were smaller than of those of frozen quiescent cells (1.03 for NAD(P)H τₘ and −1.01 for NAD(P)H α₁, respectively). We hypothesized that since thawing (increased NAD(P)H τₘ and decreased NAD(P)H α₁) and activation (decreased NAD(P)H τₘ and increased NAD(P)H α₁) induced opposite changes NAD(P)H lifetime parameters, the effects on frozen activated T cells were confounded. However, since we observed an overall similar metabolic change (increased NAD(P)H τm and decreased NAD(P)H α₁) in both frozen quiescent and frozen activated T cells within the first 4.5-hour post-thaw, our findings suggested that the effect of cryopreservation/thawing was dominant during this time window, and this potentially affected their ability to respond to activating stimulus.

### OMI Revealed Delayed Activation Response in Frozen T Cells Post-Thaw

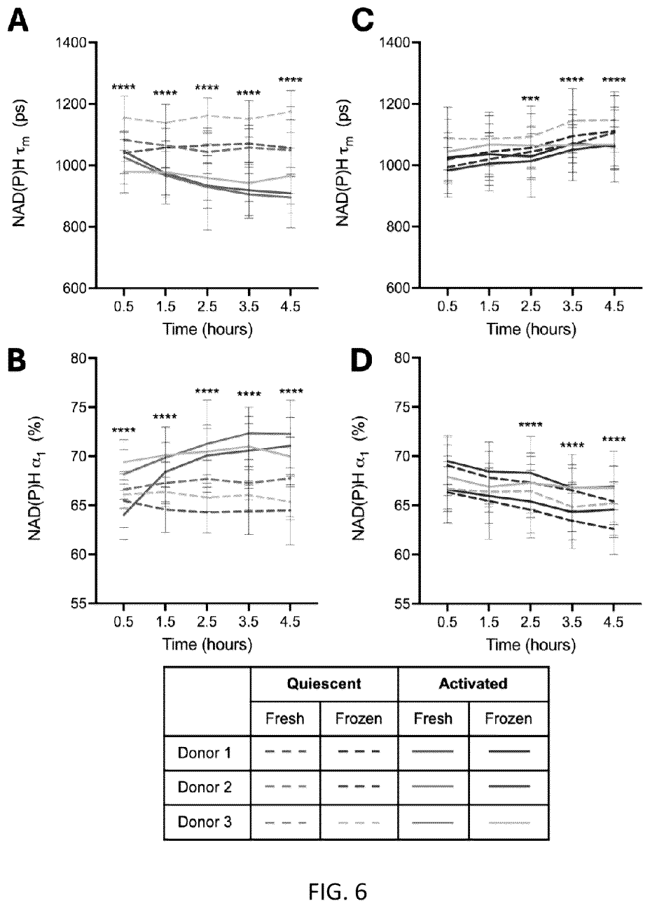

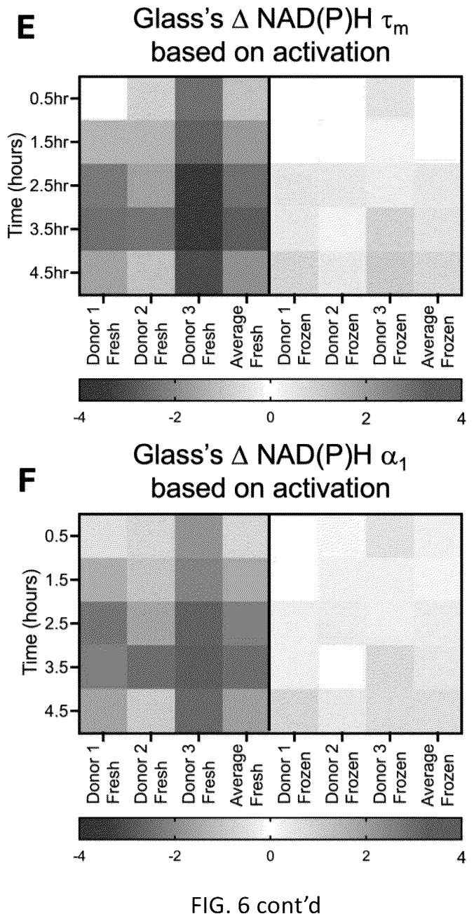

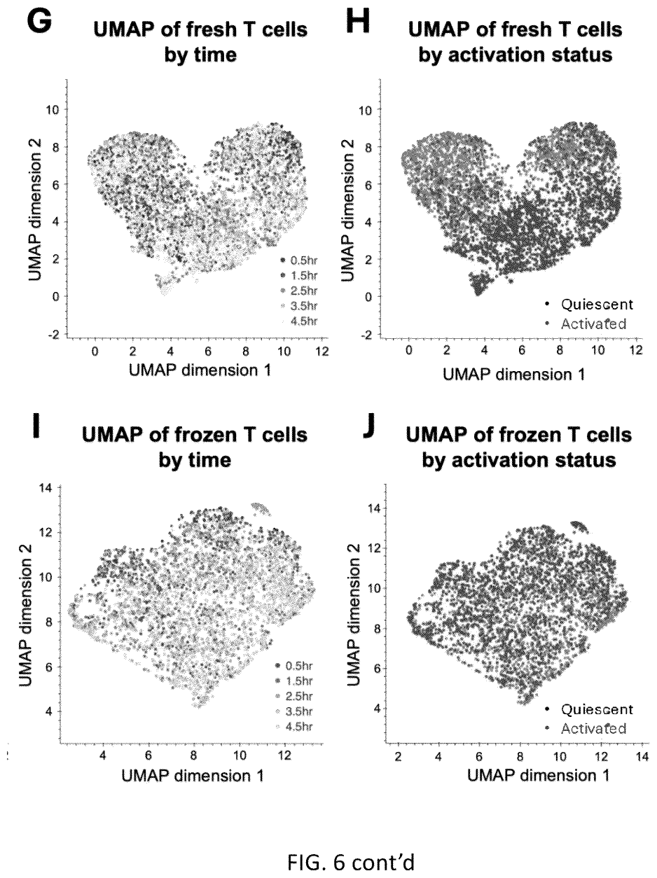

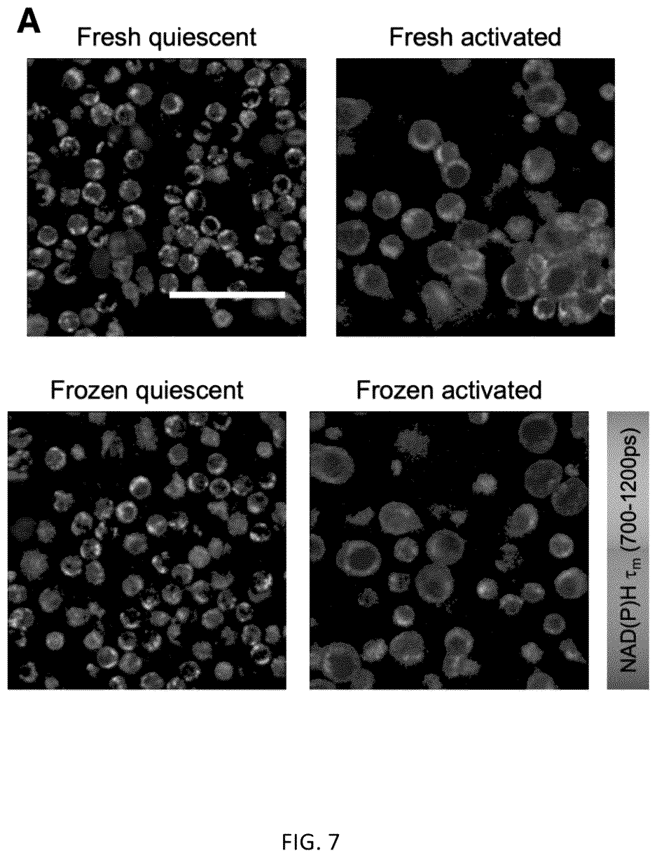

Since we observed different metabolic changes in fresh and frozen T cells upon stimulation, we further characterized the activation response in these groups compared to their donor-matched quiescent groups. We observed significantly lower NAD(P)H τₘ and higher NAD(P)H α₁ between quiescent and activated fresh T cells as early as 0.5 hour after stimulation. The differences in these NAD(P)H lifetime parameters between fresh quiescent and fresh activated T cells further widened as the stimulation duration increased (FIGS. 6A, 6B). We also observed significantly higher normalized NAD(P)H intensity in fresh stimulated T cells compared to donor-matched fresh quiescent cells (FIG. 91). The increase in NAD(P)H intensity and decrease in NAD(P)H τₘ indicated an increase in NAD(P)H abundance following activation, consistent with previous studies 33,34. We did not observe large changes in cell size due to stimulation in fresh T cells, suggesting that metabolic changes preceded morphological changes at these early time points (FIG. 9J).

Compared to fresh T cells, donor-matched frozen T cells displayed smaller and delayed changes due to stimulation, with no significant differences observed between frozen quiescent and frozen stimulated T cells until 2.5 hours post stimulation (FIGS. 6C, 6D). At the 2.5 -hour time point, frozen stimulated T cells displayed significantly lower NAD(P)H τₘ and higher NAD(P)H α₁ compared to frozen quiescent T cells. These differences continued up to 4.5-hours post stimulation (FIGS. 6C, 6D). Normalized NAD(P)H intensity and cell size of frozen T cells were less responsive to activating stimulus (FIGS. 9K, 9L).

To determine the effect size of activation on fresh and frozen T cells, we calculated the Glass's Δ at each time point with respect to donor-matched quiescent groups (FIGS. 6E, 6F). While we observed metabolic changes in a similar direction with stimulation in both fresh and frozen T cells, specifically lower NAD(P)H τₘ and higher NAD(P)H α₁, the stimulation effects on fresh T cells (FIGS. 6E, 6F—left) were consistently greater than on donor-matched frozen T cells (FIG. 6E, F—right). Activation response induced strong effects on NAD(P)H τₘ and NAD(P)H α₁ of fresh T cells, with effect sizes of up to −2.8 and 3.0 within 4.5 hours following stimulation. This indicated a difference of up to 3 standard deviations between donor-matched activated and quiescent cells. Meanwhile, for frozen T cells, activation effects on NAD(P)H τₘ and NAD(P)H ai were smaller and remained within-0.7 to 0.7. However, effect sizes due to activation did increase over time for frozen T cells, with greatest Glass's Δ observed at 4.5-hour post stimulation (FIGS. 6E, 6F—right). This suggests that as stimulation duration and time post-thaw increased, the ability of frozen T cells to respond to activating stimulus was recovered.

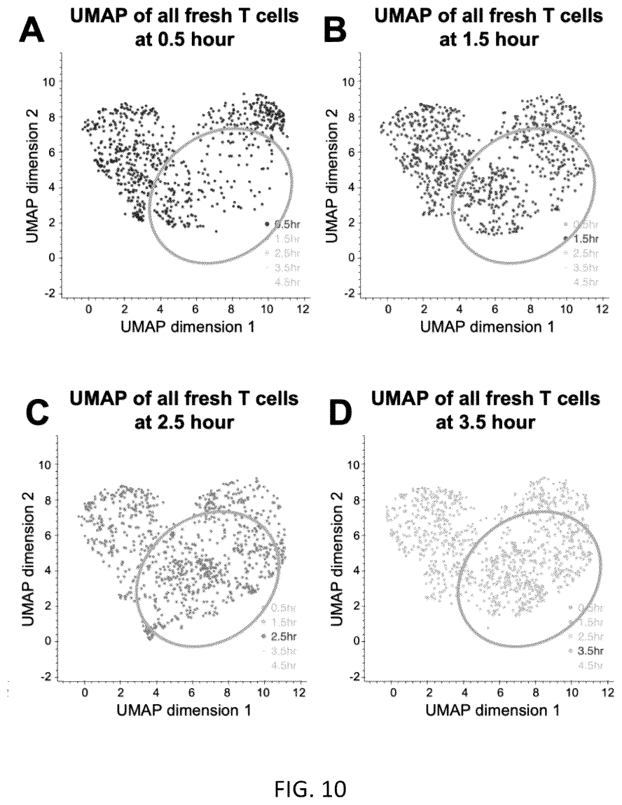

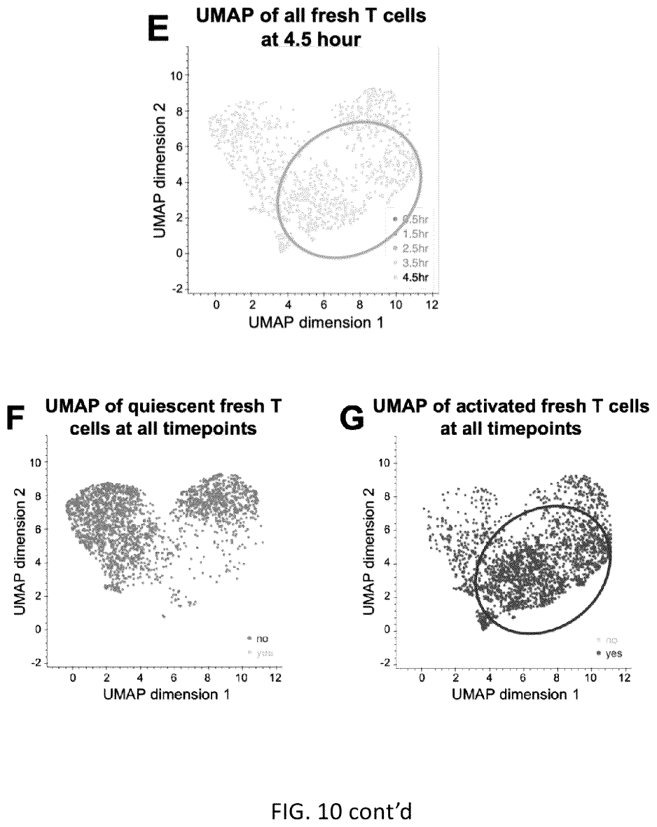

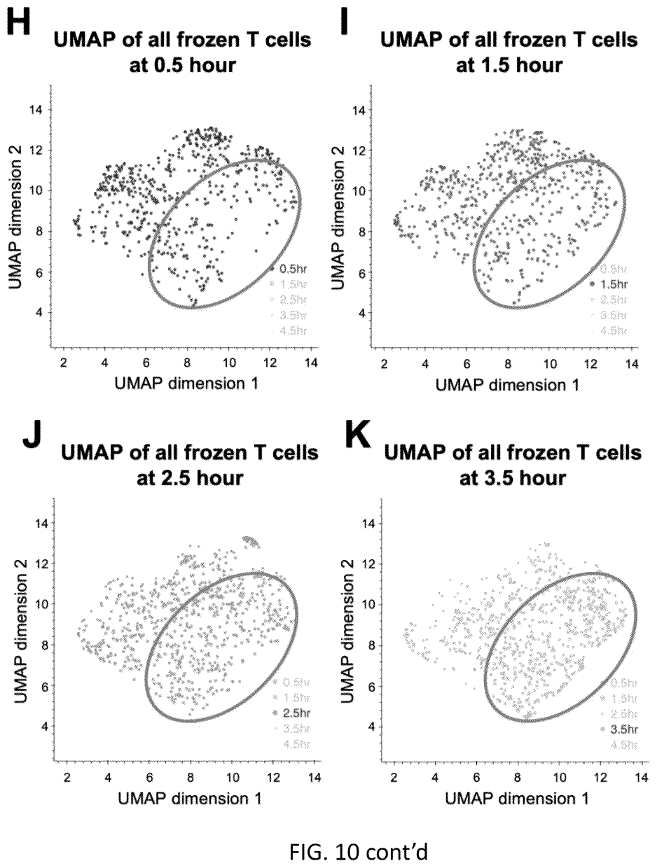

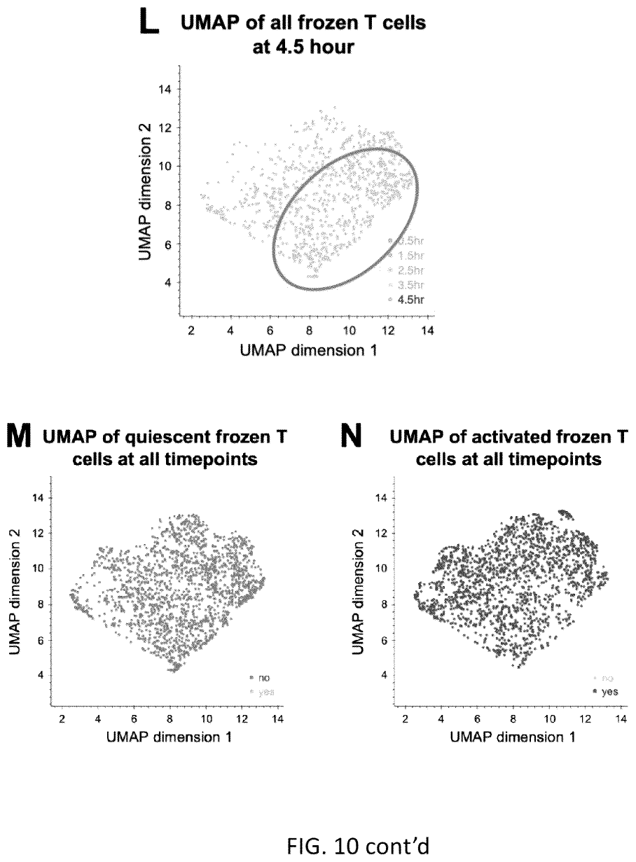

To understand the metabolic landscape of fresh and frozen T cells during activation, we projected all OMI lifetime parameters of fresh and frozen T cells with different stimulation statuses on a two-dimensional Uniform Manifold Approximation Projection (UMAP) (FIG. 6G-6J). Over time, fresh T cells moved towards bottom right corner of the UMAP (FIG. 6G, FIG. 10A-10E green circles), which aligned with the activated T cell cluster (FIG. 6H, FIG. 10G circle). This was consistent with the gradual metabolic changes in fresh activated T cells over time as reported above. We also observe distinct clusters of fresh quiescent and fresh activated T cells, suggesting distinct metabolic features of these two groups between 0.5 and 4.5 hours post stimulation (FIG. 6H, FIGS. 10F, 10G). Meanwhile, frozen T cells also moved from the top left to bottom right corner of the UMAP over the imaging time course, indicating a gradual shift in their metabolism (FIG. 6I, FIG. 10H-10L circles), consistent with the changes in OMI measurements of frozen T cells upon thawing as reported above. However, we did not observe clustering based on stimulation status (FIG. 6J, FIGS. 10M, 10N). Hence, the shift in frozen T cell metabolism between 0.5 and 4.5 hours post-stimulation was mostly governed by the impact of cryopreservation and thawing.

OMI Identified Sufficient Activation in Frozen T Cells after 48 Hours

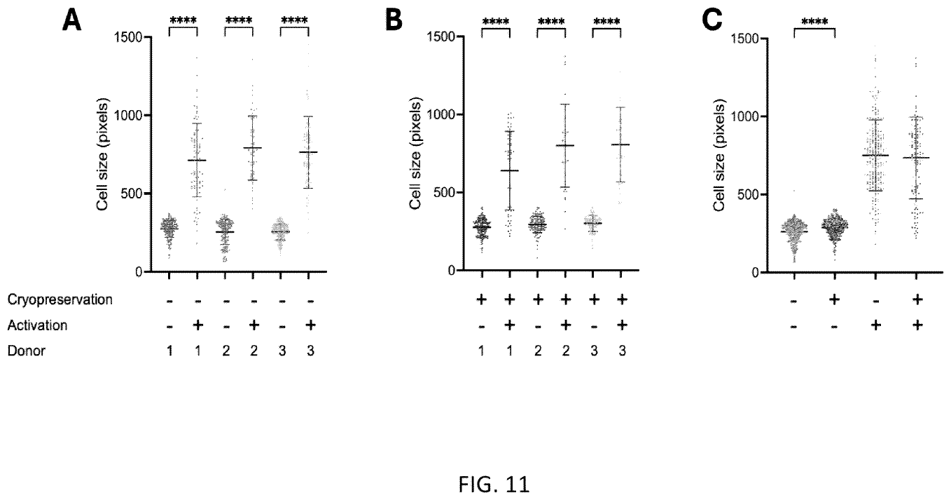

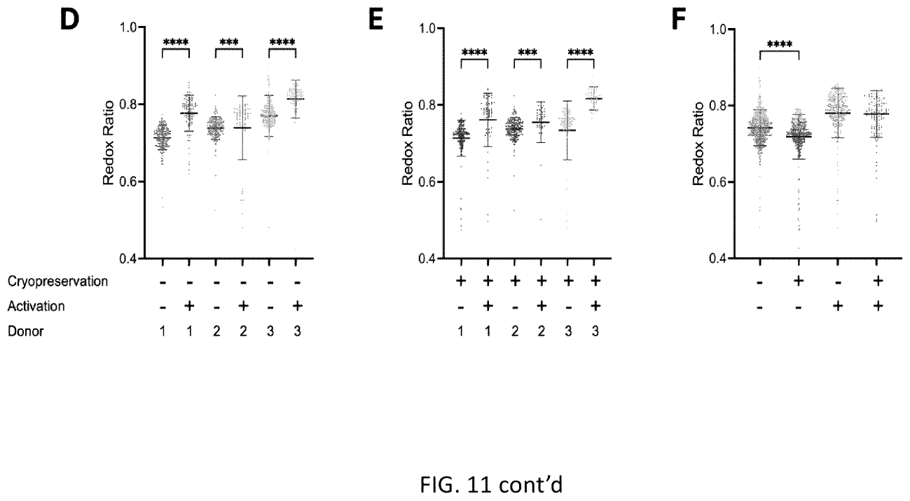

As frozen T cells showed delayed and diminished response to activating stimulus within the first 4.5 hours post-thaw, we further investigated the impacts of cryopreservation and thawing on T cell activation response at a later time point. OMI at 48 hours post stimulation revealed significant metabolic and morphological differences between quiescent and activated T cells, for both fresh and frozen groups (FIG. 7A). We observed significantly lower NAD(P)H τₘ (FIGS. 7B, 4C), higher NAD(P)H α₁ (FIGS. 7E, 7F) and greater cell size (FIGS. 11A, 11B) in fresh activated and frozen activated T cells compared to donor-matched quiescent cells. There was no significant difference in NAD(P)H τₘ between fresh and frozen cells at 48 hours; however, frozen activated T cells displayed slightly lower NAD(P)H α₁ and greater cell size compared to fresh activated T cells (FIGS. 7D, 7G, FIG. 11C). Overall, donor-matched Glass's Δ revealed consistently greater activation effects at 48 hours compared to at 4.5 hours in both fresh and frozen groups. These findings suggested that at 48 hours after thawing and stimulation, frozen T cells were able to recover their activation response. However, we did observe consistently lower fold-expansion in frozen T cells compared to their donor-matched fresh counterparts throughout a 7-day expansion period (FIG. 7H), which indicated a potential impact of cryopreservation on T cell expansion capacity.

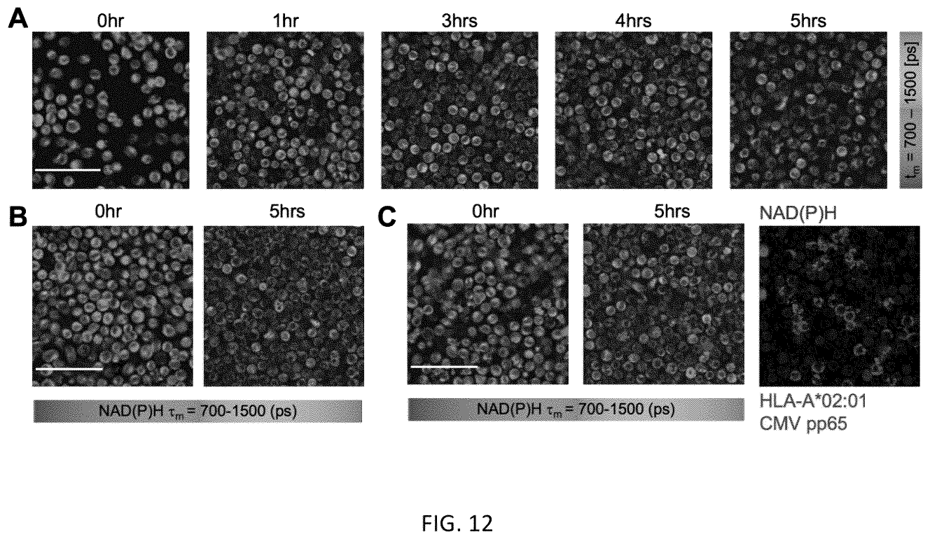

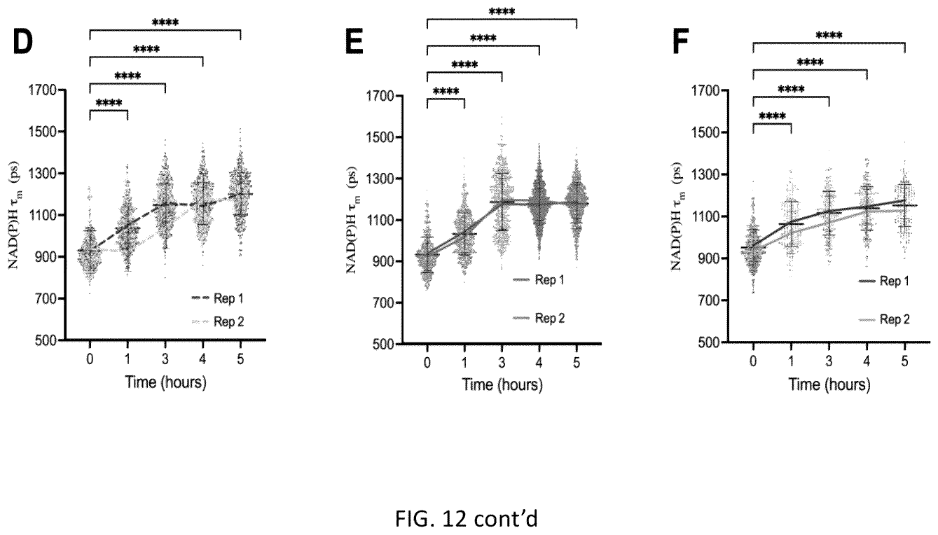

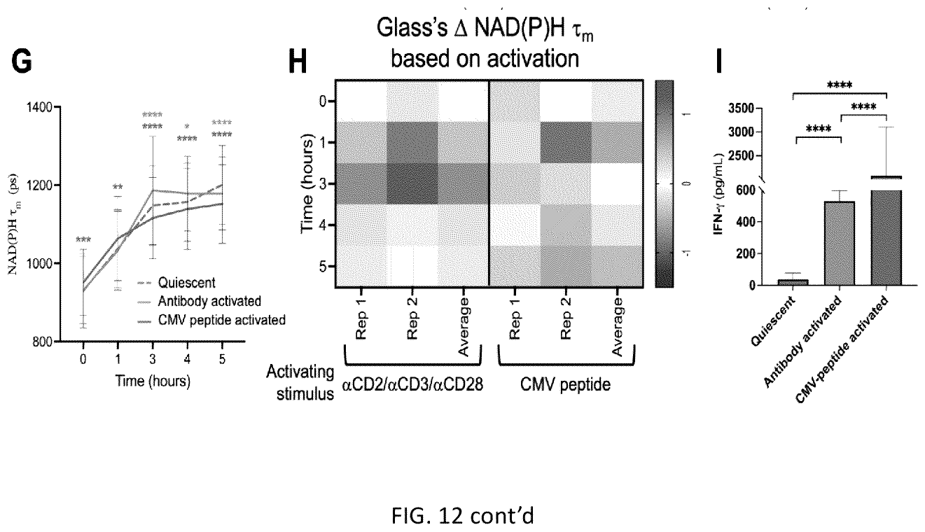

CD3/CD28 antibody is the general form of T cell stimulation, which involves binding of stimulatory and co-stimulatory receptors on T cells. Antigen-specific stimulation serves as another mode of stimulation that is relevant in several adoptive cell therapies including CAR and TCR therapies. Therefore, we also further characterized antigen-specific stimulation response in frozen T cells since cryopreservation is widely used in the context of these adoptive cell therapies. We performed OMI of CMV-specific T cells activated with StemCell αCD2/αCD3/αCD28 or HLA-matched CMV peptides. We observed similar metabolic response in frozen CMV-specific T cells upon thawing, characterized by increased in NAD(P)H τₘ that was sustained up to 5 hours post-thaw (FIG. 12A-12D). This is consistent with our data on frozen CD3 T cells reported above. Similarly, over the first 5 hours of stimulation post-thaw, CMV-specific T cells activated with activating antibody and CMV-peptide both showed significant increase in NAD(P)H τₘ (FIGS. 12E, 12F). This suggests a potential impact of cryopreservation and thawing not only on antibody-stimulation but also antigen-specific stimulation response in T cells. Notably, while low NAD(P)H τₘ is characteristic of T cell activation response, CMV-specific T cells activated with CMV peptide displayed slightly higher NAD(P)H τₘ compared to the quiescent group during the first hour post-thaw (FIG. 12G). Interestingly, compared to αCD2/αCD3/αCD28 antibody stimulation, antigen specific stimulation with HLA-matched CMV-peptide induced faster and stronger metabolic response in frozen CMV-specific T cells (FIG. 12G—pink line versus purple line, FIG. 12H—right versus left). Starting at 3 hours post-thaw, frozen CMV-specific T cells activated with HLA-matched CMV peptide showed significantly lower NAD(P)H τₘ compared to control quiescent group. This difference was sustained up to the 5-hour time point, and Glass's Δs representing antigen-specific activation effects on NAD(P)H τₘ strengthened overtime (Δ=−0.03 at 3 hours post-thaw, Δ=−0.44 at 5 hours post-thaw). Meanwhile, frozen CMV-specific T cells activated with αCD2/αCD3/αCD28 antibody did not show significantly lower NAD(P)H τₘ compared to control group until the 5-hour time point (FIG. 12G—purple line, FIG. 12H—left). As T cells produce several inflammatory cytokines such as IFN-γ upon stimulation, we further evaluated the amount of IFN-γ secreted by different CMV-specific T cells groups during the 5-hour stimulation time course. Interestingly, antigen specific stimulation with CMV peptides resulted in significantly higher IFN-γ production compared to antibody activated cells and quiescent cells (FIG. 2I). This is consistent with the greater changes in NAD(P)H τₘ observed in this group. In summary, our data suggest that cryopreservation affects both CD3 receptor stimulation and antigen-specific stimulation in T cells. However, antigen-specific stimulation induces stronger and faster metabolic as well as functional response in post-thaw T cells.

### DISCUSSION

Cryopreservation of biological samples is central in research and clinical translation of cell therapies, where the intricate manufacturing process requires transportation of cell products between the manufacturing facility and treatment site. However, cryopreservation that retains cell viability and function remains challenging for several primary immune cells such as neutrophils and natural killer cells, limiting their clinical translation. Previously, cryopreservation has been shown to impair metabolism and reduce immunomodulatory functions of mesenchymal stem cells due to heat-shock response, which potentially compromises their therapeutic benefits³⁵. Meanwhile, resting of cryopreserved natural killer cells overnight post-thaw improves their cytotoxic functions³⁶, suggesting that immediately post-thaw is a critical time window when cell function might be sensitive to cryoinjury. Since stimulation of cryopreserved cells upon thawing is involved in several stages of CAR T manufacturing and treatment, we used OMI, a label-free non-invasive imaging method, to characterize the impact of cryopreservation and thawing on T cell activation response throughout the first 4.5 hours post-thaw. Across three donors, we showed a conserved metabolic response of T cells upon thawing with high NAD(P)H τₘ and low NAD(P)H α₁ that was sustained up to 4.5 hours. These shifts in OMI measurements indicate an increase in NAD(P)H binding activity, potentially due to cell metabolism ramping up after thawing. We hypothesized that T cell metabolism upon thawing was not ready to support proper activation response. Our finding confirms a delayed, diminished activation response in frozen T cells immediately post-thaw, further supporting our hypothesis. Additionally, our data are consistent with previous studies showing impairs in immune response, specifically reduced cytokine production, due to cryopreservation in peripheral blood mononuclear cells³⁷. Interestingly, while activation response in frozen T cells was recovered at 48 hours post-thaw, their expansion capacity throughout a 7-day expansion process remained lower than donor-matched fresh T cells. This suggests that impairment in the early activation response could negatively impact T cell expansion.

Using frozen CMV-specific T cells and HLA-matched CMV-peptides, we demonstrated the impact of cryopreservation on not only CD3-mediated but also antigen-specific stimulation in T cells. However, we did observe faster and stronger metabolic changes, along with greater production of inflammatory cytokines in frozen CMV-specific T cells activated with CMV-peptide compared to CD3-antibody. Generally, anti-CD3 antibody induces cross-linking of CD3 and TCR complex to initiate an activation response. Meanwhile, antigen-specific stimulation leads to clustering of TCRs, which could result in stronger downstream signaling³⁸. Our findings indicate that OMI is sensitive to differences in T cell metabolic response towards two types of activating stimuli.

While previous research on the impact of cryopreservation on the efficacy of CAR T cell therapy suggests that cryopreserved products offer comparable potency and treatment response, there have been some evidence of lower persistence in patients receiving frozen CAR T products³⁹. This effect could be donor-dependent, or CAR-T model dependent. Cryopreservation has also been shown to impair mitochondrial coupling efficiency and ATP production in leukocytes, while increasing dependence on glycolysis⁴⁰. However, these effects were characterized after overnight resting in leukocytes with long-term cryopreservation period (up to 84 days) while our data focus on the immediate metabolic response post-thaw. Therefore, thorough characterization of T cell functional response immediately after thawing will provide insight to improve cryopreservation protocols and enhance the efficacy of CAR T cell therapy. Besides cell therapy, cryopreservation is also critical for scientific research and other medical applications (such as blood transfusion, in vitro fertilization, and testing of potential pathogens)⁴¹. Future research will further validate whether OMI is sensitive to the impacts of cryopreservation in these contexts.

### Example 2

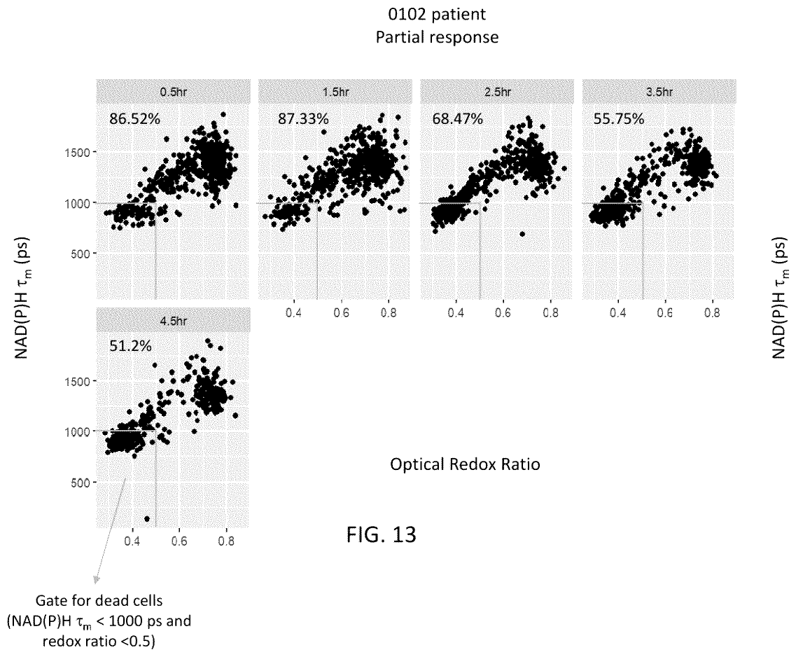

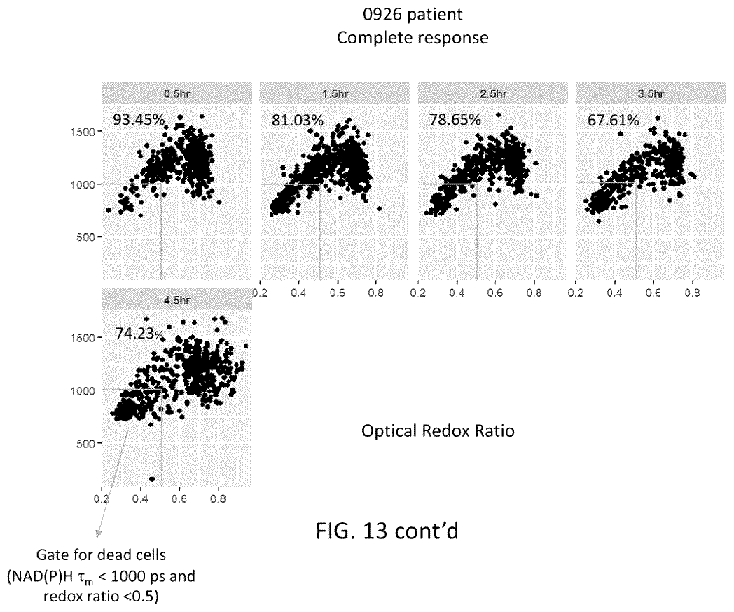

T cells from two patients with diffuse large B-cell lymphoma (DLBCL) were imaged using optical metabolic imaging (OMI) immediately after thawing. We observed a metabolic shift in these T cells, characterized by a lower NAD(P)H τₘ and a reduced optical redox ratio—opposite to what we observed in healthy T cells post-thaw. Notably, T cells with low NAD(P)H τₘ and a low redox ratio exhibited compromised membrane integrity, a hallmark of cell death (FIG. 13).

We hypothesized that this metabolic profile reflected reduced viability and compromised cell health in patient-derived T cells post-thaw. To quantify cell viability, we defined a threshold of NAD(P)H τₘ<1000 ps and an optical redox ratio <0.5 to identify dead cells. Tracking the percentage of viable cells over time revealed a progressive decline in both patient samples. Interestingly, T cells from the patient with a partial response (PR) to CAR T-cell therapy exhibited a more rapid decrease in viability compared to those from the patient with a complete response (CR), as assessed by OMI. This suggests that T cells from the CR patient may be more resilient and metabolically fit than those from the PR patient. Given that CAR T-cell therapy efficacy is influenced by T-cell health and fitness, our OMI-based post-thaw assessment could serve as a potential tool for evaluating T-cell suitability for CAR T-cell manufacturing and predicting clinical outcomes.

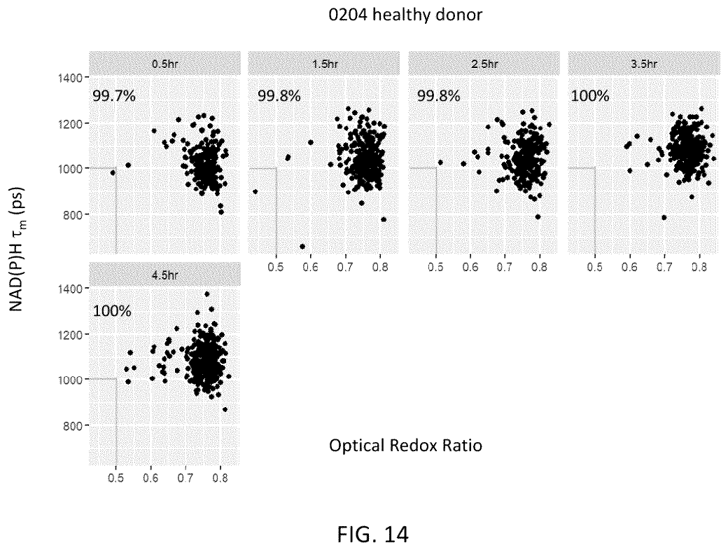

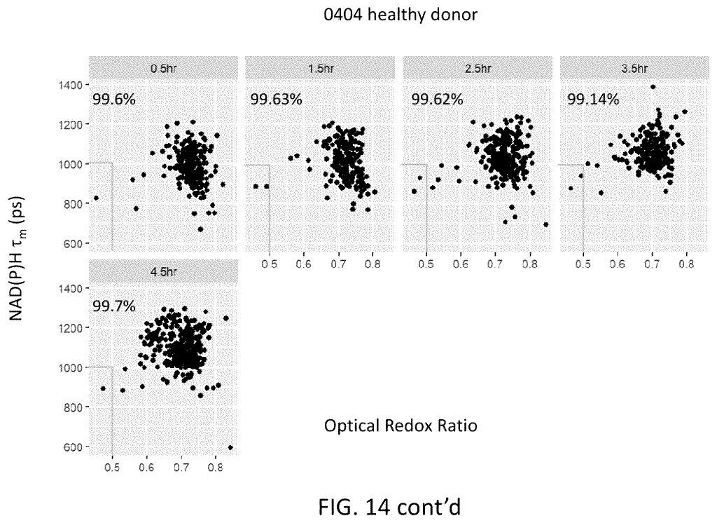

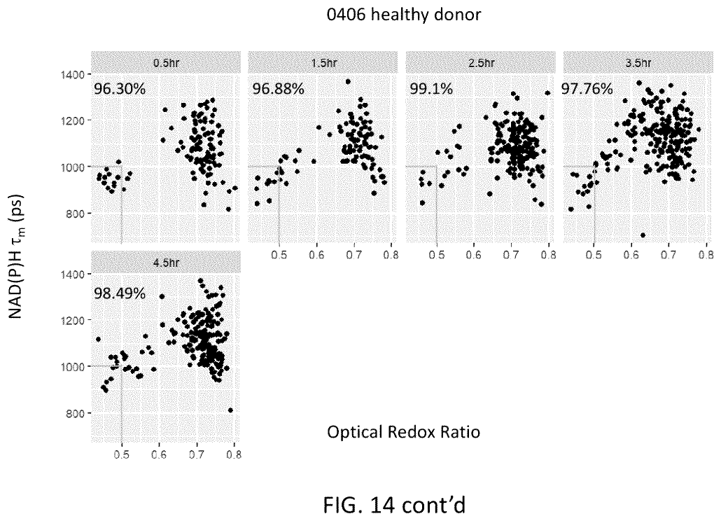

Applying the same viability threshold to cryopreserved T cells from healthy donors confirmed high post-thaw viability, further supporting the sensitivity of OMI in assessing cell health and fitness in healthy versus patient samples (FIG. 14).

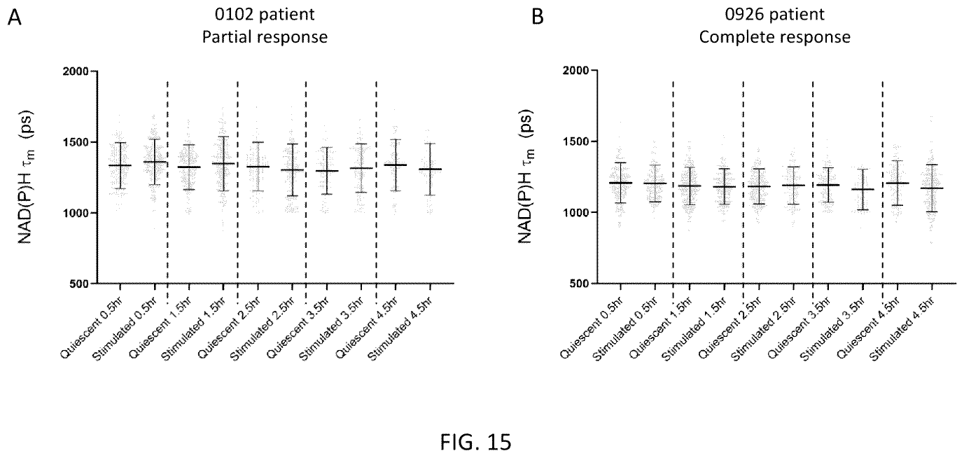

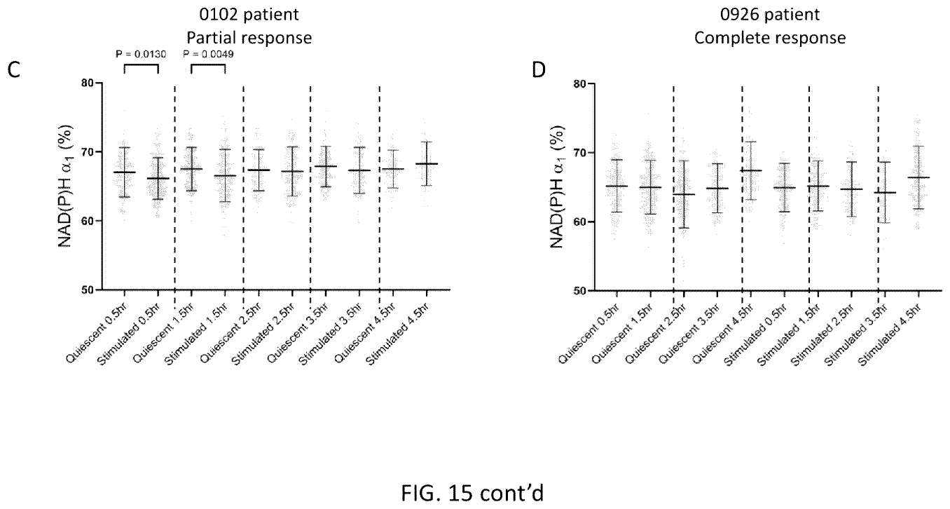

We also evaluated the activation response of cryopreserved T cells from DLBCL patients using OMI immediately post-thaw. Upon thawing, patient-derived T cells were stimulated with αCD3/αCD28 TransAct antibodies in TexMACS medium supplemented with 3% human serum and 200 U/mL IL-2. Only viable cells, as determined by the OMI viability threshold, were included in the activation response analysis (FIG. 15). While successful activation is typically associated with low NAD(P)H τₘ and high NAD(P)H α₁, these metabolic changes were absent in stimulated T cells from DLBCL patients within 4.5 hours post-thaw.

Overall, the findings indicate that cryopreserved T cells from DLBCL patients undergo significant cell death upon thawing, and even the surviving population exhibits impaired activation upon stimulation. These results highlight the potential of OMI as a sensitive tool for assessing post-thaw T-cell health and function.

References—Each of the following references is incorporated by reference in its entirety:
ADDIN ZOTERO_BIBL {“uncited”: [ ], “omitted”: [ ], “custom”: [“http://zotero.org/users/11643172/items/TRGJ79Q2”], “2. Kondo, A. T., Kerbauy, L. N., Alvarez, K. C., Oliveira, D. C., Ribeiro, A. A. F., & Prata, K. de L. (2022). Thawing and infusion of CAR-T cell products. \\uc0\\u160{ }JOURNAL OF BONE MARROW TRANSPLANTATION AND CELLULAR THERAPY, \\uc0\\u160{ }3(1), 165. https://doi.org/10.46765/2675-374X.2022v3n1p165”]]} CSL_BIBLIOGRAPHY 1. Shah, M., Krull, A., Odonnell, L., de Lima, M. J. & Bezerra, E. Promises and challenges of a decentralized CAR T-cell manufacturing model. Front. Transplant. 2, (2023).
2. Ramakrishnan, S. et al. Should we adopt an automated de-centralized model of chimeric antigen receptor-T cells manufacturing for low- and middle-income countries? A real world perspective. Front. Oncol. 12, 1062296 (2022).
3. Whaley, D. et al. Cryopreservation: An Overview of Principles and Cell-Specific Considerations. Cell Transplant. 30, 0963689721999617 (2021).
4. de Abreu Costa, L. et al. Dimethyl Sulfoxide (DMSO) Decreases Cell Proliferation and TNF-α, IFN-γ, and IL-2 Cytokines Production in Cultures of Peripheral Blood Lymphocytes. Mol. J. Synth. Chem. Nat. Prod. Chem. 22, 1789 (2017).
2. Kondo, A. T., Kerbauy, L. N., Alvarez, K. C., Oliveira, D. C., Ribeiro, A. A. F., & Prata, K. de L . . . (2022). Thawing and infusion of CAR-T cell products. JOURNAL OF BONE MARROW TRANSPLANTATION AND CELLULAR THERAPY, 3(1), 165. https://doi.org/10.46765/2675-374X.2022v3n1p165
6. Wang, X. & Rivière, I. Clinical manufacturing of CAR T cells: foundation of a promising therapy. Mol. Ther.—Oncolytics 3, (2016).
7. Ayala Ceja, M., Khericha, M., Harris, C. M., Puig-Saus, C. & Chen, Y. Y. CAR-T cell manufacturing: Major process parameters and next-generation strategies. J. Exp. Med. 221, e20230903 (2024).
8. Pearce, E. L. Metabolism in T cell activation and differentiation. Curr. Opin. Immunol. 22, 314-320 (2010).
9. Palmer, C. S., Ostrowski, M., Balderson, B., Christian, N. & Crowe, S. M. Glucose Metabolism Regulates T Cell Activation, Differentiation, and Functions. Front. Immunol. 6, (2015).
10. Soriano-Baguet, L. & Brenner, D. Metabolism and epigenetics at the heart of T cell function. Trends Immunol. 44, 231-244 (2023).
11. Zhang, X. et al. Prolonged Cryopreservation Negatively Affects Embryo Transfer Outcomes Following the Elective Freeze-All Strategy: A Multicenter Retrospective Study. Front. Endocrinol. 12, (2021).
12. Edgar, D. H., Bourne, H., Speirs, A. L. & McBain, J. C. A quantitative analysis of the impact of cryopreservation on the implantation potential of human early cleavage stage embryos. Hum. Reprod. Oxf. Engl. 15, 175-179 (2000).
13. Gualtieri, R. et al. Mitochondrial Dysfunction and Oxidative Stress Caused by Cryopreservation in Reproductive Cells. Antioxidants 10, 337 (2021).
14. Hernández-Tapia, L. G. et al. Effects of Cryopreservation on Cell Metabolic Activity and Function of Biofabricated Structures Laden with Osteoblasts. Materials 13, 1966 (2020).
15. Ostrowska, A. et al. Investigation of Functional and Morphological Integrity of Freshly Isolated and Cryopreserved Human Hepatocytes. Cell Tissue Bank. 1, 55-68 (2000).
16. Terry, C., Dhawan, A., Mitry, R. R. & Hughes, R. D. Cryopreservation of isolated human hepatocytes for transplantation: State of the art. Cryobiology 53, 149-159 (2006).
17. Miranda, P. M. et al. Human islet mass, morphology, and survival after cryopreservation using the Edmonton protocol. Islets 5, 188-195 (2013).
18. Taylor, M. J. & Baicu, S. Review of vitreous islet cryopreservation: Some practical issues and their resolution. Organogenesis 5, 155-166 (2009).
19. Cottle, C. et al. Impact of Cryopreservation and Freeze-Thawing on Therapeutic Properties of Mesenchymal Stromal/Stem Cells and Other Common Cellular Therapeutics. Curr. Stem Cell Rep. 8, 72-92 (2022).
20. Georgakoudi, I. & Quinn, K. P. Optical imaging using endogenous contrast to assess metabolic state. Annu. Rev. Biomed. Eng. 14, 351-367 (2012).
21. Skala, M. C. et al. In vivo multiphoton microscopy of NADH and FAD redox states, fluorescence lifetimes, and cellular morphology in precancerous epithelia. Proc. Natl. Acad. Sci. 104, 19494-19499 (2007).
22. Chance, B., Legallais, V. & Schoener, B. Metabolically Linked Changes in Fluorescence Emission Spectra of Cortex of Rat Brain, Kidney and Adrenal Gland. Nature 195, 1073-1075 (1962).
23. Iyanagi, T. Molecular mechanism of metabolic NAD(P)H-dependent electron-transfer systems: The role of redox cofactors. Biochim. Biophys. Acta BBA—Bioenerg. 1860, 233-258 (2019).
24. Hu, L., Wang, N., Cardona, E. & Walsh, A. J. Fluorescence intensity and lifetime redox ratios detect metabolic perturbations in T cells. Biomed. Opt. Express 11, 5674-5688 (2020).
25. Pham, D. L. et al. Development and characterization of phasor-based analysis for FLIM to evaluate the metabolic and epigenetic impact of HER2 inhibition on squamous cell carcinoma cultures. J. Biomed. Opt. 26, 106501 (2021).
26. Sharick, J. T. et al. Protein-bound NAD(P)H Lifetime is Sensitive to Multiple Fates of Glucose Carbon. Sci. Rep. 8, 5456 (2018).
27. Drozdowicz-Tomsia, K. et al. Multiphoton fluorescence lifetime imaging microscopy reveals free-to-bound NADH ratio changes associated with metabolic inhibition. J. Biomed. Opt. 19, 086016 (2014).
28. Kolenc, O. I. & Quinn, K. P. Evaluating Cell Metabolism Through Autofluorescence Imaging of NAD(P)H and FAD. Antioxid. Redox Signal. 30, 875-889 (2019).
29. Walsh, A. J. et al. Classification of T-cell activation via autofluorescence lifetime imaging. Nat. Biomed. Eng. 5, 77-88 (2021).
30. Samimi, K. et al. Time-domain single photon-excited autofluorescence lifetime for label-free detection of T cell activation. Opt. Lett. 46, 2168-2171 (2021).
31. Pham, D. L. et al. Label free metabolic imaging to enhance the efficacy of Chimeric Antigen Receptor T cell therapy. 2024.02.20.581240 Preprint at https://doi.org/10.1101/2024.02.20.581240 (2024).
32. Iritani, B. M. et al. Modulation of T-lymphocyte development, growth and cell size by the Myc antagonist and transcriptional repressor Mad1. EMBO J. 21, 4820-4830 (2002).
33. Jones, R. G. et al. Bax and Bak regulate T cell proliferation through control of ER Ca2+ homeostasis. Immunity 27, 268-280 (2007).
34. Lemire, S. et al. Natural NADH and FAD Autofluorescence as Label-Free Biomarkers for Discriminating Subtypes and Functional States of Immune Cells. Int. J. Mol. Sci. 23, 2338 (2022).
35. Galipeau, J. Concerns arising from MSC retrieval from cryostorage and effect on immune suppressive function and pharmaceutical usage in clinical trials. ISBT Sci. Ser. 8, 100-101 (2013).
36. Mata, M. M., Mahmood, F., Sowell, R. T. & Baum., L. L. Effects of cryopreservation on effector cells for antibody dependent cell-mediated cytotoxicity (ADCC) and natural killer cell (NK) activity in 51Cr-release and CD107a assays. J. Immunol. Methods 406, 1-9 (2014).
37. Martikainen, M.-V. & Roponen, M. Cryopreservation affected the levels of immune responses of PBMCs and antigen-presenting cells. Toxicol. In Vitro 67, 104918 (2020).
38. Lo, Y.-C., Edidin, M. A. & Powell, J. D. Selective Activation of Antigen-Experienced T Cells by Anti-CD3 Constrained on Nanoparticles. J. Immunol. 191, 5107-5114 (2013).
39. Dreyzin, A. et al. Cryopreserved anti-CD22 and bispecific anti-CD19/22 CAR T cells are as effective as freshly infused cells. Mol. Ther. Methods Clin. Dev. 28, 51-61 (2023).
40. Keane, K. N., Calton, E. K., Cruzat, V. F., Soares, M. J. & Newsholme, P. The impact of cryopreservation on human peripheral blood leucocyte bioenergetics. Clin. Sci. 128, 723-733 (2015).
41. Jang, T. H. et al. Cryopreservation and its clinical applications. Integr. Med. Res. 6, 12-18 (2017).

1. A method of determining a processing readiness state of a T cell, comprising:
obtaining a T cell from a subject, the T cell having been frozen and thawed;
stimulating the T cell;
measuring at least one metabolic indicator within the T cell at a plurality of timepoints;
analyzing the measurements of the at least one metabolic indicator to characterize an activation response state of the T cell at each of the plurality of timepoints; and
determining whether the T cell is ready for processing by comparing the activation response state of the T cell at each of the plurality of timepoints to a predetermined metabolic state, wherein the difference between the predetermined metabolic state and the activation response state of the T cell provides the processing readiness state of the T cell.

2. The method of claim 1, wherein stimulating the T cell comprises:
contacting the T cell with an antibody reagent or an antigen reagent.

3. The method of claim 2, wherein the antibody reagent is at least one of a soluble antibody complexes that binds to and cross-links CD3, CD28, and CD2 cell surface ligands, magnetic beads coupled with a combination of anti-human CD3 and anti-human CD28 antibodies, a polymeric matrix that contains iron oxide and humanized CD3 and CD28 agonists attached to the matrix, interleukin 2, phorbol 12-myristate 13-acetate, or ionomycin.

4. The method of claim 2, wherein the at least one metabolic indicator comprises at least one of a NAD(P)H fluorescence lifetime (τₘ, τ₁, or τ₂), a NAD(P)H fluorescence amplitude component (α₁ or α₂), a FAD fluorescence lifetime (τₘ, τ₁, τ₂), a FAD fluorescence amplitude component (α₁ or α₂), cytoplasm size, or cell size.

5. The method of claim 4, wherein measuring the at least one metabolic indicator within the T cell comprises at least one of:
obtaining a plurality of autofluorescence measurements from the T cell; or
exciting the T cell using excitation light, and
obtaining at least one of photon counts/intensity or fluorescence lifetimes from at least one of NAD(P)H or FAD excited by excitation light in the T cell.

6. The method of claim 5, wherein characterizing the activation response state of the T cell comprises:
analyzing at least one of a plurality of values of NAD(P)H τₘ, a plurality of values of NAD(P)H α₁, cytoplasm size, or cell size at each of the respective plurality of timepoints.

7. The method of claim 1, wherein the predetermined metabolic state is determined by obtaining a quiescent T cell from a subject, the quiescent T cell having been frozen and thawed;
measuring at least one metabolic indicator within the quiescent T cell at a plurality of timepoints; and
analyzing the measurements of the at least one metabolic indicator of the quiescent T cell at each of the plurality of timepoints to provide the predetermined metabolic state.

8. A system for determining a processing readiness state of a T cell comprising:
a spectrometer configured to obtain a plurality of measurements of at least one metabolic indicator within the T cell at a respective plurality of timepoints after stimulating the T cell at an initial time, the T cell having been frozen and thawed before the initial time;
a processor in electronic communication with the spectrometer; and
a non-transitory computer-readable medium accessible to the processor and having stored thereon instructions that, when executed by the processor, cause the processor to:
analyze the plurality of measurements of the at least one metabolic indicator to characterize an activation response state of the T cell at each timepoint;
compare the activation response state of the T cell at each timepoint to a predetermined metabolic state corresponding to a processing readiness state of the T cell; and
signal a user that the T cell is in the processing readiness state.

9. The system of claim 8, wherein the T cell is stimulated by contacting the T cell with at least one of an antibody reagent or an antigen reagent.

10. The system of claim 8, wherein the spectrometer, when obtaining a plurality of measurements of at least one metabolic indicator within a T cell, is configured to:
obtain a plurality of autofluorescence measurements from the T cell; or
excite the T cell using excitation light, and
obtain at least one of photon counts/intensity or fluorescence lifetimes from the T cell;
or both,
wherein the at least one metabolic indicator comprises at least one of a NAD(P)H fluorescence lifetime (τₘ, τ₁, or τ₂), a NAD(P)H fluorescence amplitude component (α₁ or α₂), a FAD fluorescence lifetime (τₘ, τ₁, τ₂), a FAD fluorescence amplitude component (α₁ or α₂), cytoplasm size, or cell size.

11. The system of claim 10, wherein the spectrometer, when exciting the T cell using excitation light, is further configured to:
excite the T cell using excitation light configured to be absorbed by at least one of NAD(P)H or FAD, and
wherein the spectrometer, when obtaining a plurality of autofluorescence measurements, is further configured to:
obtain the plurality of autofluorescence measurements from at least one of NAD(P)H or FAD stimulated by excitation light in the T cell.

12. The system of claim 11, wherein the processor, when analyzing the plurality of measurements of the metabolic indicator is further caused by the instructions to:
characterize the activation response state of the T cell based on analyzing the autofluorescence decay from the T cell at each of the respective plurality of timepoints.

13. The system of claim 12, wherein the processor is further caused by the instructions to:
obtain a plurality of values of NAD(P)H τₘ and a plurality of values of NAD(P)H α₁ at each of the respective plurality of timepoints,
analyze the plurality of values of NAD(P)H τₘ and the plurality of values of NAD(P)H α₁, and
characterize the activation response state of the T cell based on analyzing the plurality of values of NAD(P)H τₘ and the plurality of values of NAD(P)H α₁.

14. The system of claim 8, wherein the processor determines the predetermined metabolic state by:
measuring at least one metabolic indicator within a quiescent T cell at a plurality of timepoints, the quiescent T cell having been frozen and thawed; and
analyzing the measurements of the at least one metabolic indicator of the quiescent T cell at each of the plurality of timepoints to provide the predetermined metabolic state.

15. The system of claim 14, wherein the processor, is further caused by the instructions to:
identify the predetermined metabolic state based on identifying at least one of a minimum value of the plurality of values of NAD(P)H τₘ of the quiescent T cell or a maximum value of the plurality of values of NAD(P)H α₁ of the quiescent T cell.

16-20. (canceled)

21. A method of determining a processing readiness state of a T cell, comprising:
obtaining a T cell from a subject, the T cell having been frozen and thawed;
measuring at least one metabolic indicator within the T cell at a plurality of timepoints;
analyzing the measurements of the at least one metabolic indicator to characterize a health state of the T cell at each of the plurality of timepoints; and
determining whether the T cell is ready for processing by comparing the health state of the T cell at each of the plurality of timepoints to a predetermined state, wherein the difference between the predetermined state and the health state of the T cell provides a processing readiness state of the T cell.

22. The method of claim 21, wherein the at least one metabolic indicator comprises at least one of a NAD(P)H fluorescence lifetime (τₘ, τ₁, or τ₂), a NAD(P)H fluorescence amplitude component (α₁ or α₂), a FAD fluorescence lifetime (τₘ, τ₁, τ₂), a FAD fluorescence amplitude component (α₁ or α₂), cytoplasm size, or cell size, and
wherein characterizing the health state of the T cell comprises:
analyzing at least one of a plurality of values of NAD(P)H τₘ, a plurality of values of NAD(P)H α₁, cytoplasm size, or cell size at each of the respective plurality of timepoints.

23. The method of claim 21, wherein the predetermined state is determined by
obtaining a quiescent T cell from a subject, the quiescent T cell having been frozen and thawed;
measuring at least one metabolic indicator within the quiescent T cell at a plurality of timepoints; and
analyzing the measurements of the at least one metabolic indicator of the quiescent T cell at each of the plurality of timepoints to provide the predetermined state.

24. The method of claim 21, wherein the T cell comprises a representative subset of a batch of T cells.
